(19) United States
(12) Patent Application Publication
(10) Pub. No.: US 2009/0248145 A1
(43) Pub. Date: Oct. 1, 2009

(54) METHOD OF FORMING A THREE-DIMENSIONAL STRUCTURE OF UNIDIRECTIONALLY ALIGNED CELLS

(75) Inventors: Bee Eng Chan, Singapore (SG); Jie Feng, Hangzhou (CN); Jinye Shen, Singapore (CN); Vincent Chan, Singapore (SG)

Correspondence Address: SEED INTELLECTUAL PROPERTY LAW GROUP PLLC, 701 FIFTH AVE, SUITE 5400, SEATTLE, WA, 98104, US

(73) Assignee: NANYANG TECHNOLOGICAL UNIVERSITY, Singapore (SG)

(21) Appl. No.: 12/058,580

(22) Filed: Mar. 28, 2008

(51) Int. Cl. A61F 2/06; C12N 5/06

(52) U.S. Cl. 623/1.41; 435/378

(57) ABSTRACT

The present invention provides a method of forming a three-dimensional structure of unidirectionally aligned cells. The method comprises providing a substrate with a microchannel. The microchannel is defined by at least a pair of opposing lateral walls and a base. In at least a portion of the microchannel the distance between the pair of opposing lateral walls is within the micrometer range. A first plurality of cells is seeded in the microchannel and the cells are allowed to proliferate up to at least a density of at least 90%. Thereby contact guidance cues are provided by the pair of opposing lateral walls of the microchannel, such that the cells align unidirectionally. Thereby a first layer of aligned cells is also formed at the base of the microchannel. A second plurality of cells is seeded in the microchannel, which already comprises a first layer of aligned cells. When allowing cells of the second plurality of cells to proliferate up to at least substantial confluence, contact guidance cues are again provided by the lateral walls of the microchannel. The cells also align unidirectionally and form a second layer of aligned cells.

### FIELD OF THE INVENTION

The present invention relates generally to the field of tissue engineering, and therein to a method of forming a three-dimensional structure of unidirectionally aligned cells.

### BACKGROUND OF THE INVENTION

The major aspect of tissue engineering is the design and fabrication of constructs for the replacement of nonfunctional tissue. For example vascular disease, a major cause of death worldwide commonly caused by cardiovascular disorder, aneurysmal disease or hypertension require the use of bypass grafting. For this technique an autologous graft or synthetic prostheses is necessary. In many patients, the availability of suitable native arteries/veins are limited especially if the patients have undergone multiple operations. Furthermore, synthetic prostheses using polyester or expanded polytetrafluoroethylene for small diameter (<3-4 mm diameter) bypass grafts have so far only shown limited success. The lack of functional small diameter grafts with long term patency has made the development of tissue engineered blood vessels an urgent research priority. Presently however, tissue-engineered grafts often fail to fulfill their role in vivo, for instance due to inappropriate burst strengths and compliance mismatch to tissue, e.g. blood vessels, of the host.

A replacement tissue should mimic the three-dimensional (3D) microarchitecture of cells and extracellular matrix of the respective native tissue. Tissues containing a smooth muscle component, such as vascular tissues, the esophagus or the intestines possess well-aligned smooth muscle cells. For example in native blood vessels, the medial layer provides the main structural support of the vessel (i.e. strength, elasticity, and contractility) and consists of multiple layers of circumferentially aligned vascular smooth muscle cells (SMCs). This layered and aligned 3D cellular microstructure is thought to have a close correlation with vessel structural integrity and vasoactivity and thus the recapitulation of this native SMC 3D microstructure in vitro is one main challenge in vascular tissue engineering.

A general method of engineering tissues in vitro is the use of a scaffold, in or on which cells are grown. A respective scaffold should be biodegradable before or after implantation. Simply culturing cells on a scaffold provides however no means of controlling cellular organization (FIG. 1A). In contrast thereto, cells are highly organized in vivo (FIG. 1B). Further, cell morphology of SMCs in vivo is spindle-shaped. Such contractile SMCs, aligned in circumferential direction in a vessel allow for vessel wall constriction or dilatation upon cell contraction or relaxation. Upon in vitro culture, this morphology of SMCs is lost, cells show a fibroblast-like, synthetic phenotype (for further details see e.g. Stegemann, J. P. & Nerem, R. M., Experimental Cell Research (2003) 283, 146-155, incorporated herein by reference in its entirety).

Two general approaches have so far been followed to align SMCs in order to be able to provide tubular-structured tissues. One approach is based on the influence of mechanical stimulation (Jeong, S. I., et al., Biomaterials (2005) 26, 1405-1411) and requires the use of a porous scaffold. Myofibroblasts, which can differentiate into a smooth muscle cells, have likewise been shown to be suitable cells for this approach (Cha, J. M., et al., Artificial Organs (2006) 30, 4, 250-258). This approach can be performed in two dimensional culture using a porous sheet-type substrate (Cha et al., 2006, supra). To be performed in a three dimensional culture, this approach poses high requirements in terms of elasticity and tensile strength on the scaffold used, thereby highly restricting the materials suitable for scaffold formation (see Jeong et al., 2006, supra). Furthermore, cells need to be injected into the scaffold to achieve three dimensional culture (ibid.), posing additional experimental challenges.

A second approach is based on the orientation of SMCs in microchannels via contact guidance (Glawe, J. D., et al., Journal of Biomedical Materials Research, Part A (2005) 75A, 1, 106-114, incorporated herein by reference in its entirety). Microchannels of 60 μm and 20 μm width caused cell alignment, accompanied by an alignment of actin filaments and nuclei (ibid.). The narrower the channels the higher was the alignment observed. Stegemann & Nerem have shown that this approach can also be performed in a three dimensional collagen gel (Stegemann & Nerem, 2003, supra). However, upon doing so a markedly decreased proliferation and also α-actin expression of SMCs was observed (ibid.). This suppression in 3D gels was explained as an effect of “steric hindrance” caused by the presence of the collagen matrix, such that cells did not have sufficient space to divide. Also, in another long term experiment, Seliktar et al. found that exogenous collagen disappears quickly in the earlier stage of culture period (8 days) leading to low vessel strength, possibly due to increased matrix metalloproteinase-2 (MMP-2) activated by mechanical stimulation (Seliktar, D., et al., Tissue Engineering (2003) 9, 657).

The second approach has also been demonstrated for fibroblasts (Norman, J. J. & Desai, T. A., Tissue Engineering (2005) 11, 3/4, 378-386). By embedding fibroblasts within a collagen gel and then dispensing them into deep microchannels, Norman et al. (ibid.) have created multilayered, elongated and aligned fibroblasts. Their three-dimensional scaffold did not, however, result in a three-dimensional culture as the fibroblasts in the collagen matrix grew mostly along the walls of the channels, resulting in, effectively, a two-dimensional culture, albeit with certain three-dimensional aspects.

Accordingly, so far no simple method for generating well aligned and uniform distribution of cells in three dimensional culture has been identified that can easily be carried out.

It is therefore an objective of the present invention to provide a method of tissue engineering a three-dimensional structure of unidirectionally aligned cells that overcomes some of the above explained difficulties.

### SUMMARY OF THE INVENTION

According to a first aspect of the present invention, a method of forming a three-dimensional structure of unidirectionally aligned cells is provided. The method includes providing a substrate with a microchannel. This microchannel is defined by at least a pair of opposing lateral walls and a base. The microchannel has a distance between the pair of opposing lateral walls, which is—in at least a portion of the microchannel—within the micrometer range. The method also includes seeding a first plurality of cells in the microchannel. The method further includes allowing cells of the first plurality of cells to proliferate up to at least substantial confluence at the base of the microchannel. Thereby firstly contact guidance cues are provided by the pair of opposing lateral walls of the microchannel. As a result the cells align unidirectionally. Secondly, thereby a first layer of aligned cells of the first plurality of cells is formed at the base of the microchannel. The method also includes seeding a second plurality of cells in the microchannel. The base of the microchannel already comprises a first layer of aligned cells of the first plurality of cells (see above). Further the method includes allowing cells of the second plurality of cells to proliferate up to at least substantial confluence at the base of the microchannel. Thereby firstly contact guidance cues are provided by the lateral walls of the microchannel. As a result the cells align unidirectionally. Secondly, thereby a second layer of aligned cells of the second plurality of cells is formed at the base of the microchannel.

The method of the invention allows the buildup of multiple layers of aligned and confluent cells. As each cell layer contains cells aligned in a desired direction—with many cell types components of the cytoskeleton of the respective cells align—the method of the invention typically allows forming a tissue engineered construct with improved tensile and contractile strength, which is particularly desirable in engineered vasculature. Furthermore, the method of the invention is rapid since it can be carried out with high seeding density. The method of the invention is therefore particularly useful in fabricating multi-layered structures within a period of time that is short enough for the needs of clinical therapeutic applications.

In a second aspect the invention provides a method of forming a vascular graft. The method includes providing a biodegradable substrate with a plurality of microchannels. Each of the plurality of microchannels is defined by at least a pair of opposing lateral walls and a base. In at least a portion of each microchannel the distance between the pair of opposing lateral walls is within the micrometer range. Each of the microchannels has at least one common lateral wall with a further microchannel of the plurality of microchannels. Each microchannel thus shares at least one common lateral wall with a further microchannel. This common lateral wall separates the two microchannels. The method also includes seeding a first plurality of cells in the plurality of microchannels. Further, the method includes allowing cells of the first plurality of cells to proliferate up to at least substantial confluence at the bases of the plurality of microchannels. Thereby firstly contact guidance cues are provided by the pair of opposing lateral walls of each microchannel. As a result, the cells align unidirectionally. Secondly, thereby a first layer of aligned cells of the first plurality of cells is formed at the base of each microchannel. The method also includes seeding a second plurality of cells in each microchannel. The base of each microchannel already comprises a first layer of aligned cells of the first plurality of cells (see above). Further, the method includes allowing cells of the second plurality of cells to proliferate up to at least substantial confluence at the base of the microchannel. Thereby firstly contact guidance cues are provided by the pair of opposing lateral walls of each microchannel. As a result the cells align unidirectionally. Secondly, thereby a second layer of aligned cells of the second plurality of cells is formed at the base of each microchannel.

In a third aspect the invention provides a method of treating a patient in need of vascular prosthesis. The method includes replacing a diseased or damaged portion of the patient's vasculature with the vascular graft obtained according to the method of the second aspect. Such a vascular graft may, for example, be used to bypass obstructions to blood flow caused by the presence of atherosclerotic plaques. As a further example, it may be used in providing arterial-venous shunts in dialysis patients or in treating aneurysms.

### BRIEF DESCRIPTION OF THE DRAWINGS

The invention will be better understood with reference to the detailed description when considered in conjunction with the non-limiting examples and the accompanying drawings.

FIG. 1A depicts the three-dimensional orientation of cells in a conventional scaffolding technique. FIG. 1B depicts a typical vascular tissue (left), also in magnification (right), illustrating the organization of smooth muscle cells (32) into unidirectionally aligned layers.

FIG. 2 depicts an embodiment of pulsatile perfusion for culturing smooth muscle cells (B) after being seeded into a porous scaffold (A).

FIG. 3 depicts a further embodiment of pulsatile perfusion, in which a porous sheet is provided (A), on which smooth muscle cells are grown under pulsatile flow (B).

FIG. 4 shows another technique of obtaining unidirectionally aligned smooth muscle cells (Shen, J. Y, et al., Tissue Engineering (2006) 12, 8, 2229-2240). A substrate with a microchannel (A) or a plurality thereof (C) is provided, in which cells are seeded and grown (B and D, respectively).

FIG. 5 is a simplified flow diagram of the method of fabricating the device of the invention.

FIG. 6 depicts a substrate with microchannels used in an embodiment of the invention in top view (FIG. 6A) and cross-sectional view (FIG. 6B).

FIG. 7 depicts an overview of an embodiment of the method according to the present invention. After cells (1) have been seeded in a microchannel (5) with lateral walls (3) and a base (4), cells are allowed to reach confluence (I). A second plurality of cells (1) is seeded (II), allowed to reach confluence (III), and this procedure may be repeated several times.

FIG. 8 depicts a further embodiment of the method according to the present invention. Cells (1) are seeded in a microchannel (5) with lateral walls (3) and a base (4), and allowed to reach confluence (I). A biodegradable material is added into the microchannel (II), a second plurality of cells (1) seeded in the microchannel (III), and the procedure repeated several times, if desired.

FIG. 9 depicts an embodiment of a method of the invention, in which a substrate (6) with a circumferential wall is provided (A), on which a plurality of microchannels (5), separated by walls (3), is arranged. Layers of cells are formed according to the present invention (see e.g. FIG. 7 or FIG. 8) in the microchannels (B).

FIG. 10 depicts a further embodiment of a method of the invention, in which a flat substrate (7) of biodegradable, bendable material with a plurality of microchannels (5), separated by walls (3), is provided (A). Layers of cells are formed according to the present invention (see above (B), and the substrate is bent (C), thereby forming a circumferential wall of a tubular structure (D).

FIG. 11 depicts an embodiment of forming a vascular graft that resembles the embodiment depicted in FIG. 10. However, the microchannels (5) of the provided substrate (A), in which layers of cells are grown (B) stretch perpendicular to the long side of the substrate (7). Upon bending (C), the longitudinal axis of microchannels is distorted, so that the microchannels become circular, surrounding the lumen (11) formed (D).

FIG. 12 illustrates the 4 most common folding directions (I-IV) with respect to the microchannels (5), in which cells (1) are grown.

FIG. 13 illustrates schematically a folded tubular structure suitable for smooth muscle cells (cells not shown). The microchannels (5), separated by walls (3), revolve around the central lumen (11).

FIG. 14 shows the correlation between the seeding density of smooth muscle cells and the time needed for the cells to reach confluence in microchannels, when grown according to the method of the present invention.

FIG. 15 illustrates the formation of a confluent layer of smooth muscle cells, at the beginning of culture (A) vs. cells at confluence (B), in a microchannel in a method according to the present invention.

FIG. 16 depicts the alignment of smooth muscle cells grown on microchannels of a substrate according to the present invention, as shown in FIG. 8, on the two vertical walls of the microchannel.

FIG. 17 depicts F-actin-staining of confluent smooth muscle cells, grown in a microchannel (“C”) with lateral walls (“W”) in a method according to the present invention, as shown in FIG. 8, (A), compared to control cells grown on a uniformly flat surface (B).

FIG. 18 depicts different layers of F-actin-stained smooth muscle cells seeded and grown on microchannels of a substrate according to the method of the invention, as shown in FIG. 8 (A: bottom layer; B: second layer; C: third layer; D: top layer).

FIG. 19 depicts alpha-actin-stained smooth muscle cells seeded and grown on microchannels of a substrate according to the present invention, as shown in FIG. 8, (A), compared to cells on a gel layer overfilled above the top of the microchannel walls (B).

FIG. 20 is a cross-sectional view of four layers of F-actin-stained smooth muscle cells, seeded and grown according the method of the present invention.

FIG. 21 depicts F-actin-stained smooth muscle cells, seeded and grown on microchannels of a substrate according to the present invention, as shown in FIG. 7 (every other of five layers shown: A: top layer; B: central, third, layer; C: bottom layer; D: 3D composite image of A, B, and C in top view; E: 3D composite image of A, B, and C viewed from the bottom of a channel).

### DETAILED DESCRIPTION OF THE INVENTION

The phenomena of contact guidance is known to be a factor that orients cells in vivo. The contact guidance based approach to align SMCs (see above) has been further developed by the present inventors (Shen, J. Y., et al., Tissue Engineering (2006) 12, 8, 2229-2240, incorporated herein by reference in its entirety; US patent application 2007/009572 A1, incorporated herein by reference in its entirety). In two dimensional culture, SMCs grow with spindle-shaped morphology in narrow microchannels of a width of 10 μm and 25 μm, albeit to much lower cell density than on a flat surface (Shen et al., 2006, supra). In wider microchannels of a width of 80 μm to 160 μm, SMCs initially show fibroblast morphology with random orientation, and switch to elongated morphology with unidirectional orientation when nearing confluence. Cells in such wide microchannels grow to similar density as on flat surfaces (Shen et al., 2006, supra).

Upon doing so the applicants made the surprising finding that multiple layers of cells could be grown on a top of each other, which allows the formation of a three-dimensional structure of cells. If desired, a biodegradable material may be used to separate the cell layers.

Any substrate may be used in the method of the invention as long as it provides a physically permissive contact guidance structure. Generally such a physically permissive contact guidance structure can be taken to be or include one or more microchannels of desired dimensions (see below). The substrate may include or be of any desired material. Illustrative examples of suitable material are a metal, a metalloid, ceramics, a metal oxide, a metalloid oxide, oxide ceramics, carbon or a polymer (such as an elastomer). Examples of suitable metalloids include, but are not limited to silicon, boron, germanium, antimony and composites thereof. Examples of suitable metals include, but are not limited to iron (e.g. steel), aluminum, gold, silver, chromium, tin, copper, titanium, zinc, aluminum, lead and composites thereof. A respective oxide of any of these metalloids and metals may be used as a metalloid oxide or metal oxide respectively. As an illustrative example, the base substrate may be of quartz or a glass. Examples of ceramics include, but are not limited to, silicate ceramics, oxide ceramics, carbide ceramics or nitride ceramics.

If desired, the substrate may include or be of translucent material such as, glass, quartz or a plastic material. Suitable plastic materials include, but are not limited to, polymethylmethacrylates (e.g. polymethyl-methacrylate (PMMA) or carbazole based methacrylates and dimethacrylates), polystyrene, polycarbonate, and polycyclic olefins. A further illustrative example of a material that is additionally suitable for the generation of a substrate that allows light to pass only to a certain extent is fluoro-ethylene-propylene (FEP). In some embodiments the substrate includes or consists of a biodegradable material, such as a biodegradable polymer. A biodegradable material is readily susceptible to biological processing in vivo. It can be degraded by a living organism or a part thereof (e.g., bacterial or enzymatic action) or by the impact of the ambience, such as exposure to light, moisture, elevated temperature and/or air. Degradation of a biodegradable material may result in the formation of primary degradation products such as compounds of low molecular weight, which then decay further through the action of a living organism. In the context of the present invention the term “biodegradable material” particularly refers to matter that can be completely removed from a localized area, by physiological metabolic processes. A “biodegradable” compound can, when taken up by a cell, be broken down into components by cellular machinery such as lysosomes or by hydrolysis that the cells can either reuse or dispose of without significant toxic effect on the cells. Examples of biodegradation processes include enzymatic and non-enzymatic hydrolysis, oxidation and reduction. Suitable conditions for non-enzymatic hydrolysis, for example, include exposure of biodegradable material to water at a temperature and a pH of a lysosome (i.e. the intracellular organelle). The degradation fragments typically induce no or little organ or cell overload or pathological processes caused by such overload or other adverse effects in vivo.

Various examples of biodegradable materials are known in the art, any of which are generally suitable for use in the method of the present invention. As some illustrations of polymers that are considered to be biodegradable may serve: a polyglycolide, a polylactide, a polycaprolactone, a polyamide, a biodegradable aliphatic polyester, and/or copolymers thereof, with and without additives (e.g. calcium phosphate glass), and/or other copolymers (e.g. poly(caprolactone lactide), a poly(ester amide), a poly(amino acid), a pseudo-poly(amino acid) such as a poly(iminocarbonate-amide)copolymer, poly(lactide glycolide), poly(lactic acid ethylene glycol)), poly(ethylene glycol), poly(ethylene glycol)diacrylate, a polyalkylene succinate, polybutylene diglycolate, a polyhydroxybutyrate, polyhydroxyvalerate, a polyhydroxybutyrate/polyhydroxyvalerate copolymer; poly(hydroxybutyrate-co-valerate); a polyhydroxyalkaoates, a poly(caprolactone-polyethylene glycol) copolymer, poly(valerolactone), a polyanhydride, a poly(orthoester) and/or a blend with a polyanhydride, poly(anhydride-co-imide), an aliphatic polycarbonate, a poly(propylene carbonate), a poly(hydroxyl-ester), a polydioxanone, a polyanhydride ester, a polycyanoacrylate, a poly(alkyl cyanoacrylate), a poly(amino acid), a poly-(phosphazene), a poly(propylene fumarate), poly(propylene fumarate-co-ethylene glycol), a poly(fumarate anhydride), fibrinogen, fibrin, gelatin, cellulose, a cellulose derivative, chitosan, a chitosan derivative such as chitosan NOCC, chitosan NOOC-G or NO-carboxy-methyl chitosan NOCC, alginate, a polysaccharide, starch, amylase, collagen, a polycarboxylic acid, a poly(ethyl ester-co-carboxylate carbonate), poly(iminocarbonate), poly(bisphenol A-iminocarbonate), poly(trimethylene carbonate), poly(ethylene oxide), poly(epsilon-caprolactone-dimethyltrimethylene carbonate), a poly(alkylene oxalate), poly(alkylcarbonate), poly(adipic anhydride), a nylon copolyamide, carboxymethyl cellulose; copoly(ether-esters) such as a PEO/PLA dextran, a biodegradable polyester, a biodegradable polyether, a polydihydropyran, a biodegradable polyketal such as poly (hydroxylmethylethylene di(hydroxymethyl)ketal) or poly[1-hydroxymethyl-1-(2-hydroxy-1-hydroxymethyl-ethoxy)-ethylene oxide], a polydepsipeptide, a polyarylate(L-tyrosine-derived) and/or a free acid polyarylate, a poly(propylene fumarate-co-ethylene glycol) such as a fumarate anhydride, a hyaluronate, poly-p-dioxanone, a polyphosphoester, polyphosphoester urethane, a polysaccharide, starch, rayon, rayon triacetate, latex, and/or copolymers, blends, and composites of any of the above.

The substrate may be of any geometry. It may for instance have one circumferential wall or a plurality of lateral walls. It may also have a base and a top wall. If desired, it may include any geometrical element such as a recess, a dent, a bulge, a step, a ledge, an extrusion, or any combination thereof. The microchannel may be located anywhere on the substrate. Where a plurality of microchannels is provided on the substrate, they may be located at any position relative to each other and in any orientation with respect to each other. In some embodiments at least some microchannels of a plurality of microchannels run parallel to each other. Such parallel microchannels may for instance be adjacent or contiguous to each other. In one embodiment adjacent microchannels share a common lateral wall. In some embodiments the microchannel includes a branching.

The microchannel is defined by at least a pair of opposing lateral walls and a base, such as base wall. The distance between the two opposing lateral walls of at least one portion of the microchannel (or of the entire microchannel) is within the micrometer range. As used herein, the term micrometer range refers to a range of between 1 μm and 1000 μm. The walls of the microchannel may be of any desired internal surface characteristics and any desired material as long as they allow cells of a desired type to grow therein. Furthermore, different internal areas of the walls of the microchannel may provide different surface characteristics and include or consist of different materials.

In typical embodiments the microchannel is open along its length in that it defines a trench (as opposed to a sub-surface channel). The microchannel may span (e.g. laterally, diagonally etc.) the entire length/width of the substrate. Where the microchannel does not span the entire length/width of the substrate it may further be bounded by an additional pair of lateral walls, which are typically in opposing relationship. The term “opposing relationship” refers to the direction of matter that could flow through the recess and/or the channel, such as an axis of the channel. Accordingly, the two lateral walls of the filler member may be arranged in any angle with respect to each other, as long as the first and the second aperture are not facing the same direction. The first and the second lateral wall may for instance be inclined with respect to each other in an angle from 0 to 90°.

The microchannel may have any desired shape, including straight, bent or meandering (or otherwise winding) and may include one or more bends, kinks or branches as long as the distance between the two opposing lateral walls of at least one portion of the microchannel is within the micrometer range. In typical embodiments the microchannel has one longitudinal axis. The microchannel may possess a transverse section of any desired profile, such as being a cuboid (e.g. with rectangular or square shaped profile) or alternatively a hemi-sphere or any other suitable irregular profile. The profile of the channel may also change its shape along the length of the microchannel, for instance gradually or stepwise. The lateral walls may also be of any surface topology. Any of them may for instance be a curved wall, a stepped wall or a straight wall. In some embodiments the surface of a microchannel may include a patterned surface, for example including one or more elements of a groove, a gouge, a dent or a bulge, which may be of micro- and/or nanometer-scale and which may thus also be much smaller than the cells that are desired to be used. Any number of elements of such pattern may also provide selected surface properties. Such a pattern may for example be obtained using a combination of spin-coating, electron-beam lithography, sputtering, lift-off and covalent coupling with a selected compound as described by Goto et al. (Anal. Bioanal. Chem. (2008) 390, 817-823).

The microchannel may be of any length. Regardless of the shape and profile, the microchannel has in some embodiments a depth/width ratio (also known as “aspect ratio”) of up to about 15, up to about 25, up to about 30, up to about 40 or up to about 50. Where a hemispherical shaped microchannel is formed in the substrate, it is to be noted that the microchannel is then defined by a continuous wall, which includes wall portions that can be defined as a base and as two opposing lateral walls. The same may apply to an irregularly shaped microchannel. In some embodiments the distance between the two opposing lateral walls of at least a portion of the microchannel or of the entire microchannel is selected to be in the range from about 10 μm to about 800 μm or to about 600 μm, such as about 10 μm (or about 20 μm) to about 500 μm, about 10 μm to about 200 μm, about 200 μm to about 300 μm or about 50 μm to about 500 μm. The exact distance in the respective portion or entire microchannel ought to be selected depending on the cell type intended to be grown in the microchannel, as different optimum ranges exist for different cells. The desired range can conveniently be optimized in single-layer experiments. As an illustrative example, the present applicants have found ranges from about 10 μm to about 200 μm, such as e.g. about 60 μm to about 200 μm, well suited for growing cell line smooth muscle cells. For primary smooth muscle cells they found ranges from about 50 μm to about 400 μm, such as e.g. about 60 μm to about 400 μm or about 50 μm to about 300 μm, well suited. In some embodiments the distance between the two opposing lateral walls is at least essentially uniform throughout at least a portion of the microchannel. In this portion the distance of the two opposing lateral walls may be selected to be in the above range.

The pair of opposing lateral walls may be arranged at a uniform distance throughout the entire length of the microchannel. In other embodiments the distance across the width of the microchannel may vary along the length thereof. Accordingly, a variation in the distance between the pair of opposing lateral walls may be taken to be a change in diameter of a respective microchannel. The lateral walls of the microchannel may be designed as surfaces of the substrate in which the microchannel may for instance form a cavity or trench. The term “wall” may in such embodiments be understood as referring to a two-dimensional area, which may be of any desired geometric properties. The lateral walls of the microchannel may also form three-dimensional structures such as a cuboid or a cube. In such embodiments the wall possesses a transverse section with a profile, such as a rectangular, a square shaped, a circular, an oval profile, or any other desired irregular profile. The profile of such a wall may also change its shape along the length of the wall, for instance gradually or stepwise. In such embodiments the term “wall” may be understood as referring to a three-dimensional body that also provides a barrier, likewise defining the margins of the microchannel. A respective wall may include or consist of the same matter as the (remaining) substrate or be of a different matter (see above for examples). Embodiments in which the lateral walls define a three-dimensional structure may typically be desired in embodiments where a plurality of microchannels is arranged in the substrate. If two or more microchannels, or portions thereof, are contiguous to each other they may share a common wall. A wall that is shared by two contiguous microchannels may be of any width. As an illustrative example, the width may be in the range, but not limited thereto, of from about 1 μm to about 100 μm, such as e.g. about 5 μm, about 10 μm, about 25 μm or about 50 μm.

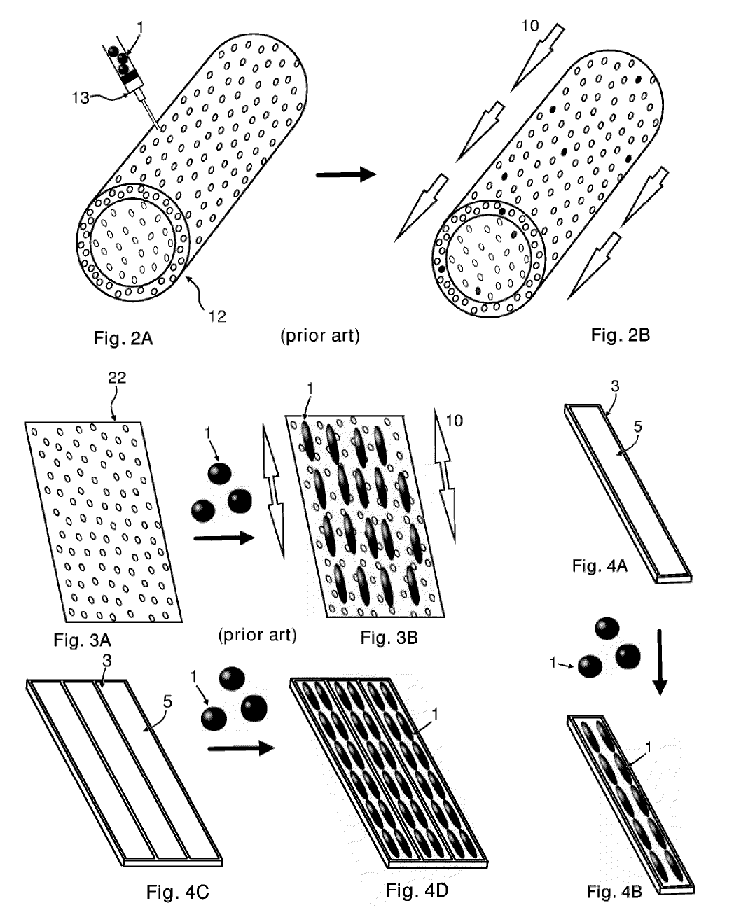

The microchannel may include further elements, for example elements that assist in providing guidance cues. As an illustrative example a 3D scaffold of aligned fibrils of collagen or a proteoglycan may be formed magnetically as described by Torbet et al. (Biomaterials (2007) 28, 4268-4276). Where desired, such a 3D scaffold of aligned fibrils may include regions in which fibrils are aligned in different orientations, e.g. in form of lamellae (ibid., FIG. 2 thereof).

The base of the microchannel may be of any desired geometrical properties and internal surface characteristics. It may be arcuate, include one or more steps, dents, inversions, bulges, grooves or striations. It may also include portions that are inclined to any desired extent, including an entirely inclined base. In some embodiments it may be of uniform topology, for example at least essentially flat or at least essentially bent at a uniform angle. In some embodiments a portion of the base is at least essentially complanate, including having an at least essentially straight surface. A respective portion or portions of the base may cover any percentage of the base, including the entire base. In some embodiments the respective portion or portions of the base corresponds to the portion of the microchannel, in which the distance between the two opposing lateral walls is selected in the above range, e.g. the range from about 10 μm to about 500 μm.

As noted above, the substrate may be of any desired surface properties. However, at least the base of the microchannel(s) ought to have a surface, to which selected cells can adhere. For this purpose the surface properties of the base of the microchannel, the entire channel or the entire substrate that includes the microchannel(s) may be altered where required. The respective surface, or a part thereof, may for instance be altered by means of a treatment carried out to alter characteristics of the solid surface. Such a treatment may include various means, such as mechanical, thermal, electrical or chemical means. As an illustrative example, the surface properties of any hydrophobic surface can be rendered hydrophilic by coating with a hydrophilic polymer or by treatment with surfactants. Examples of a chemical surface treatment include, but are not limited to exposure to hexamethyldisilazane, trimethylchlorosilane, dimethyldichlorosilane, propyltrichlorosilane, tetraethoxysilane, glycidoxypropyltrimethoxy silane, 3-aminopropyltriethoxysilane, 2-(3,4-epoxy cyclohexyl)ethyltrimethoxysilane, 3-(2,3-epoxy propoxyl)propyltrimethoxysilane, polydimethylsiloxane (PDMS), γ-(3,4-epoxycyclohexyl)ethyltrimethoxysilane, poly(methyl methacrylate) or a polymethacrylate co-polymer, urethane, polyurethane, fluoropolyacrylate, poly(methoxy polyethylene glycol methacrylate), poly(dimethyl acrylamide), poly[N-(2-hydroxypropyl)methacrylamide] (PHPMA), α-phosphorylcholine-o-(N,N-diethyldithiocarbamyl)undecyl oligoDMAAm-oligo-STblock co-oligomer (cf. e.g. Matsuda, T., et al., Biomaterials, (2003), 24, 4517-4527), poly(3,4-epoxy-1-butene), 3,4-epoxy-cyclohexylmethylmethacrylate, 2,2-bis[4-(2,3-epoxy propoxy)phenyl]propane, 3,4-epoxy-cyclohexylmethylacrylate, (3′,4′-epoxycyclohexylmethyl)-3,4-epoxycyclohexyl carboxylate, di-(3,4-epoxycyclohexylmethyl)adipate, bisphenol A (2,2-bis-(p-(2,3-epoxy propoxy)phenyl)propane) or 2,3-epoxy-1-propanol. A surface treatment may also include applying a coating of a peptide, a polypeptide or a protein (such as a cell surface protein), for instance fibronection, vitronectin, laminin, collagen, gelatine, polylysine or the synthetic peptides arginine-glycine-aspartate (RGD) and tyrosine-isoleucine-glycine-serine-arginine (YIGSR). A large number of biodegradable polymers, such as poly(glycolic acid), poly(L-lactic acid) or poly(lactic-co-glycolic acid) (see also above) are also known to be suitable for a surface treatment to facilitate cell adhesion.

A first plurality of cells is seeded in the microchannel, or in any desired number of microchannels of a plurality of microchannels. The cells may be seeded by any means. They may for example be dispensed on a top of the microchannel(s), e.g. by means of a pipette. Any cell may generally be selected to be seeded into the microchannel. However, in the method of the invention cells are selected that can align unidirectionally. Examples of respective cells include, but are not limited to, smooth muscle cells, skeletal muscle cells, endothelial cells, stem cells, progenitor cells, myocytes, bone marrow cells, neurons, pericytes and fibroblasts. Cells used in the method of the present invention may be of any source. They may for example be native cells, including cells isolated from tissue, or they may be cells of a cell line. Respective cells may also be modified, e.g. treated by an enzyme, exposed to radiation, transformed by the incorporation of heterologous matter (an organelle, genetic material, inorganic matter etc.), or they may be recombinant or transgenic. The cells may be seeded at any density, as long as they are able to proliferate on the base of the microchannel(s) and as long as they are seeded below confluence. Seeding densities as low as 1×10⁴ cells/cm² were found suitable for smooth muscle cells.

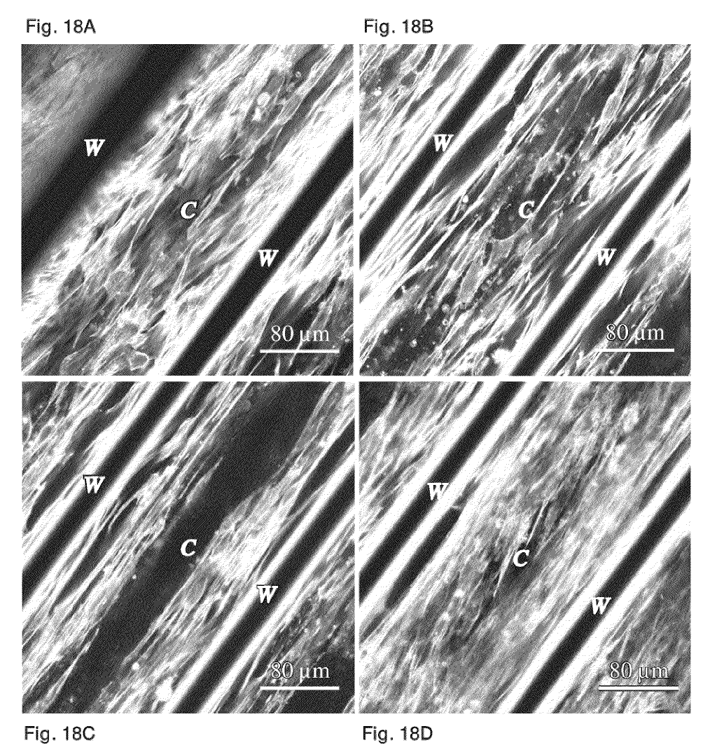

The cells are allowed to adhere to the base of the microchannel and to proliferate. Depending on the cells used, they are allowed to proliferate up to a density of about 80% to about 100%, such as about 85%, 90% or 95%. The cells are allowed to proliferate near to confluence or, in other words, allowed to reach, at least substantially, confluence at the base of the microchannel. The term “confluence” is used herein—unless stated otherwise—in the regular meaning to describe a state in which cells have grown within a certain amount of space. At this point surface to surface contact with other cells causes the individual cells to inhibit their growth. In line with this meaning of the term “confluence”, the expression “proliferate near to confluence” or “reach at least substantially confluence means that the majority of cells (say for example, 70%, 80%, 90%, 95%, 98% or 99% of the cells) have grown to an extent that this majority of cells contact at least one neighboring cell such that the growth of the individual cells of the majority of cells is inhibited by contact inhibition. This means that is not necessary that all cells only a majority of the cells proliferate to confluence. In the method of the invention during proliferation the cells align unidirectionally. Smooth muscle cells show an aligned and elongated morphology in a direction that is at least essentially parallel to the closest lateral wall of the channel, which is typically the direction that corresponds to the length of the microchannel(s). Depending on the cell type, the contact guidance and/or mechanical cues provided by the walls of the microchannel(s) cause in many embodiments the unidirectionally aligned cells to show an at least essentially unidirectional orientation of actin filaments (see e.g. FIG. 18 or FIG. 19). Accordingly, by pre-selecting a design of the microchannel, and in particular the walls thereof, a “tissue axis” may be defined. This may be particularly helpful in embodiments where a tissue graft is to be formed from the cells and/or a biodegradable substrate. This two-dimensional alignment and elongation occurs in the presence of the channel wall and does not require any further factors. The channel walls provide contact guidance cues and/or mechanical cues, thereby causing the cells to align, and in a number of embodiments to show an elongated morphology.

In the method of the invention a first layer of aligned cells has been formed (see above). A second plurality of cells is then seeded in the microchannel. The cells of the second plurality of cells may be of the same type, including the same batch or from the same source, as the cells of the first plurality of cells. The second plurality of cells may also include or consist of cells that are different—whether in terms of source, batch, type, etc—from the first plurality of cells. The cells of the second plurality of cells may be seeded directly on the first layer of aligned cells of the first plurality of cells at the base of the microchannel. In this case the cells of the first and the second plurality of cells contact each other in the microchannel.

In some embodiments the first layer of aligned cells of the first plurality of cells at the base of the microchannel is covered with a first layer of a biodegradable material such as collagen (see above for examples). Any biodegradable material may be used, as long as its use is not detrimental to the viability of the cells of the first plurality of cells at the channel base. The biodegradable material may be allowed to form an at least essential straight, e.g. an at least essentially planar, surface. The biodegradable material may in some embodiments form a hydrogel, in which the cells of the first plurality of cells are encapsulated. In embodiments where a biodegradable material is used to cover the first cell layer of aligned cells, the second plurality of cells is seeded on the respective biodegradable material.

The cells of the second plurality of cells are allowed to adhere to the cells, the biodegradable material or other matter on base of the microchannel, and to proliferate near to confluence. Depending on the cells used, they are allowed to proliferate up to a density of about 80% to about 100%, such as about 85%, 90% or 95%. Similar to the cells of the first plurality of cells, the cells of the second plurality of cells are allowed to reach, at least substantially, confluence at the base of the microchannel. Also, the cells align unidirectionally during proliferation (see above for the morphology of SMCs). This two-dimensional alignment occurs in the presence of the channel walls, which provide mechanical and/or contact guidance cues, as indicated above. Again, the presence of the channel walls may induce a morphology change in the second plurality of cells (see above). Similar to the first plurality of cells, it may also cause a unidirectional alignment of actin filaments in the unidirectionally aligned cells. A second layer of aligned cells of the second plurality of cells is formed at the base of the microchannel.

The method of the invention may further include any desired repetition of seeding a further plurality of further cells (different or corresponding to the first and/or the second plurality of cells) and allowing the cells to adhere and to proliferate up to at least substantial confluence as indicated above. Any type of cells may be used that can be unidirectionally aligned, such as smooth muscle cells, skeletal muscle cells, endothelial cells, stem cells, progenitor cells, myocytes, bone marrow cells, neurons, pericytes or fibroblasts. Any of the cells used, including the cells of the first or the second plurality of cells, may be human cells. As long as the further cells (besides the first and the second plurality of cells) that are seeded are included in the microchannel(s), they are exposed to the mechanical and/or contact guidance cues provided by the channel walls. Thereby further layers of aligned cells are formed. As an illustrative example the number of cell layers may be selected from about 2 to about 10, such as 3, 4, 5, 6, 7, 8, 9 or 10 cell layers. Where desired, a biodegradable material (see above) may be deposited into the channel before seeding a plurality of cells, thereby covering the previously formed cell layer (see above).

As explained above, the substrate may include a plurality of microchannels with each channel being defined by at least a pair of opposing lateral walls and a base. Independent for each channel, in at least a portion of the individual microchannels the distance between the two opposing lateral walls is from about 10 μm to about 600 μm, such as about 25 μm to about 400 μm, 50 μm to about 300 μm or about 50 μm to about 450 μm. In some embodiments at least two microchannels of the plurality of microchannels are separated by a common wall. In some embodiments each of the plurality of microchannels shares at least one of its lateral walls with another microchannel. As noted above, a wall separating two microchannels may of any desired thickness, i.e. width. In some embodiments the thickness is at least essentially constant along the length of the wall. In other embodiments the thickness of a wall of one or more of the microchannels varies along the length of the respective microchannel.

In some embodiments a common wall of two microchannels, in one embodiment each wall separating contiguous microchannels, has a thickness from about 0.5 μm to about 200 μm, such as from about 0.5 μm to about 100 μm, about 10 μm to about 200 μm, about 1 μm to about 100 μm or about 1 μm to about 50 μm. The walls of the microchannels are of an independently selected material. In some embodiments all lateral channel walls are of the same material. This material is or includes in some embodiments the same material as the base. In one embodiment it is or includes the same material as the material of the substrate.

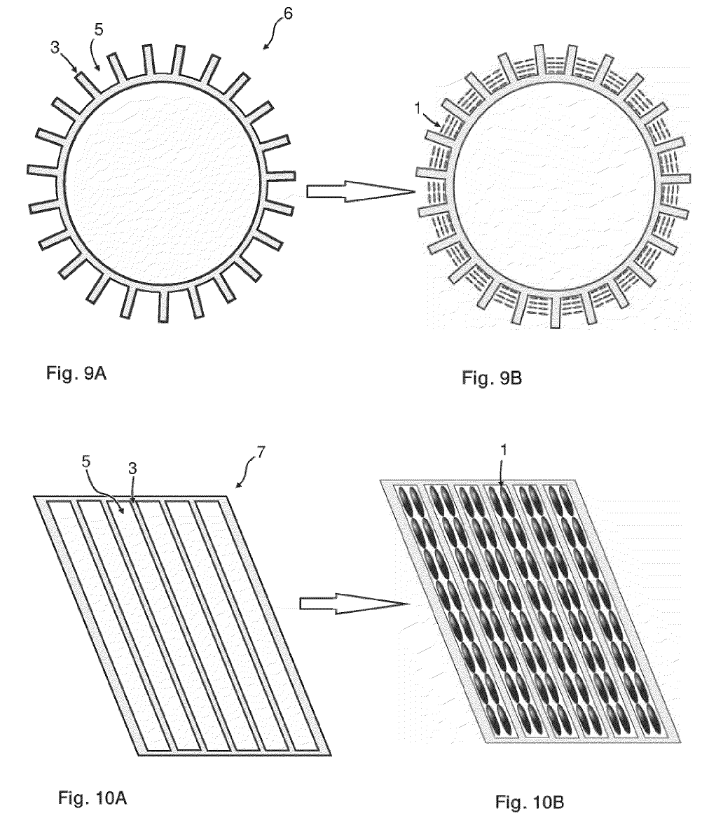

In some embodiments the common wall separating (any or all) two microchannels of the plurality of microchannels includes or is of a biodegradable material. In such embodiments the method of the invention may further include allowing the respective common wall separating two microchannels of the plurality of microchannels to be degraded. Thereby a wider channel is formed. In some embodiments the substrate includes or is defined by a circumferential wall. On this circumferential wall there is arranged the plurality of microchannels. Both the substrate and the channel walls may include or consist of a biodegradable material such as a biodegradable polymer. During the method of the invention the microchannels are filled by layers of aligned cells, as described above. The method may then include degrading the substrate and the channel walls, thereby providing a three-dimensional structure of unidirectionally aligned cells. The substrate may for instance have/mimic the shape of an organ such as a vessel. It may for instance furthermore be hollow. In such an embodiment the outer surface of the substrate may include a plurality of channels, as depicted in FIG. 9. If the substrate is hollow, it may provide a circumferential wall (cf. FIG. 9), surrounding a lumen. In such an embodiment the substrate may provide a plurality of microchannels, both on its inner surface, facing the lumen, and on its outer surface, facing the ambience. Layers of different cell types may be formed in the microchannels on a respective inner and outer surface of such a substrate. The cells may be selected to correspond to the location of cell types of a respective organ, e.g. vessel—in that cells may for instance be grown on the inner surface of the substrate that are found facing the organ lumen. Cells that are located in e.g. deeper layers of the organ, not facing the lumen, may then be grown on the outer surface of the substrate. According to the in vivo alignment of the selected cells in an organ, e.g. a vessel, the orientation of the microchannels on the inner and on the outer surface of a respective substrate may be selected.

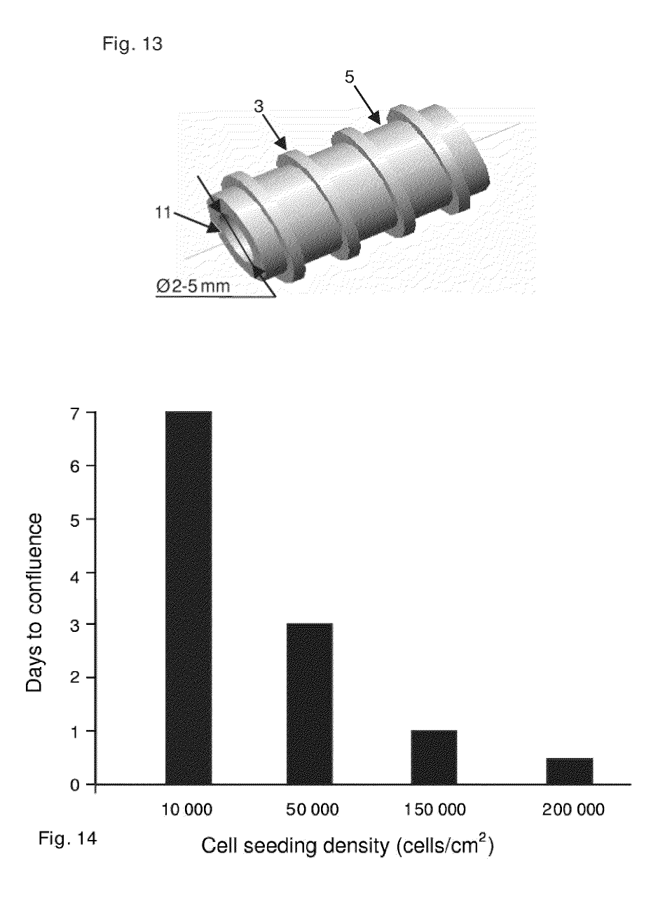

As an illustrative example, endothelial cells face the lumen of a blood vessel, where they are aligned in the longitudinal direction of the vessel, i.e. in the direction corresponding to the length of the vessel. Smooth muscle cells are found in the media, i.e. a deeper layer of the blood vessel, below the endothel. The smooth muscle cells are aligned in the circumferential direction of the vessel, i.e. in a circular manner surrounding the vessel (cf. FIG. 13). The method of the present invention may be used to form a three-dimensional structure that is a graft such as a vascular graft. As used herein, the terms “implant”, “graft”, and “tissue graft” are used interchangeably, referring to homologous or heterologous tissue or a cell group, or matter such as an artificial material, which is inserted into a particular site of a body and thereafter forms a part of the body. A synthetic tissue or three-dimensional structure formed by the method of the present invention can accordingly be used as an implant. Examples of conventional grafts include, but are not limited to, organs or portions of organs, blood vessels, blood vessel-like tissue, heart, cardiac valves, pericardia, dura matter, joint capsule, bone, cartilage, cornea, tooth, and the like. Therefore, grafts encompass any one of these which is inserted into an injured part so as to compensate for the lost portion. Grafts include, but are not limited to, autografts, allografts, and xenografts, which depend on the type of their donor. As used herein, the term “autograft” (a tissue, a cell, an organ, etc.) refers to a graft (a tissue, a cell, an organ, etc.) which is implanted into the same individual from which the graft is derived. As used herein, the term “autograft” (a tissue, a cell, an organ, etc.) may encompass a graft from a genetically identical individual (e.g. an identical twin) in a broad sense. As used herein, the terms “autologous” and “derived from a subject” are used interchangeably. Therefore, the term “not derived from a subject” in relation to a graft indicates that the graft is not autologous (i.e., heterologous). An “allograft” is a graft (a tissue, a cell, an organ, etc.) which is transplanted from a donor genetically different from, though of the same species, as the recipient. Since an allograft is genetically different from the recipient, the allograft (a tissue, a cell, an organ, etc.) may elicit an immune reaction in the recipient. Examples of such grafts (a tissue, a cell, an organ, etc.) include, but are not limited to, grafts derived from parents (a tissue, a cell, an organ, etc.). A “xenograft” is a graft (a tissue, a cell, an organ, etc.) which is implanted from a different species. Therefore, for example, when a human is a recipient, a porcine-derived graft (a tissue, a cell, an organ, etc.) is called a xenograft (a tissue, a cell, an organ, etc.). The “recipient” (acceptor) is an individual that receives a graft (a tissue, a cell, an organ, etc.) or implanted matter (a tissue, a cell, an organ, etc.) and is also called “host”. An individual providing a graft (a tissue, a cell, an organ, etc.) or implanted matter (a tissue, a cell, an organ, etc.) is called “donor” (provider).

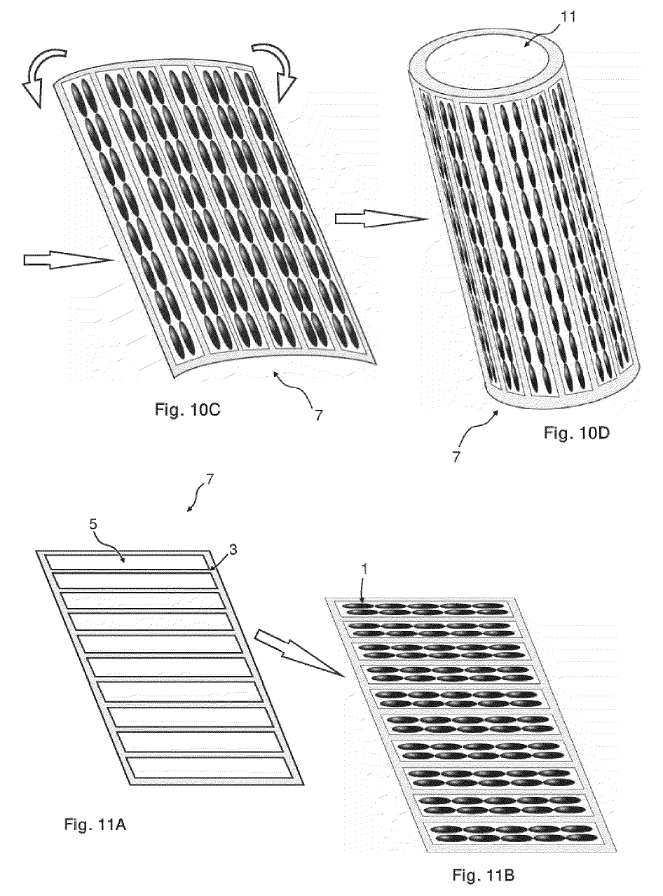

In embodiments of the invention where a vascular graft is formed, a tubular substrate may be used for forming such a graft. A first plurality of microchannels may be provided on the outside of the tubular substrate, facing the ambience. A second plurality may be provided on the inside of the tubular substrate, facing the lumen. It may then be desired to form layers of smooth muscle cells in the first plurality of cells, and to form layers of endothelial cells in the second plurality of cells, in order to mimic the layering of cells found in blood vessels. To provide contact guidance cues/mechanical cues that cause both cells types to align in the same direction as corresponding native cells of a blood vessel, a perpendicular orientation of the first and the second plurality of microchannels could be selected. The first plurality of microchannels could run along the length of the lumen of the tubular substrate (cf. FIG. 10D, showing such microchannels on the outer side of a tubular substrate). The second plurality of microchannels could be orientated in a circumferential manner, running around the longitudinal axis of the tubular substrate as depicted in FIG. 13. It is recalled that not only a substrate of cylindrical or tubular shape can be used for forming a graft, but that besides this illustrative example any other desired geometric form may be selected.

As noted above, the substrate used in the method of the invention may in some embodiments be degraded to any desired extent during or after the method of the invention, as long as a three-dimensional structure of unidirectionally aligned cells has already been formed and is allowed to remain at least essentially intact. Accordingly the method of the invention allows for the formation of scaffold-free implants and a respective scaffold-free implanting method. Using the method of the invention it is thus possible to avoid problems arising from the scaffold such as contamination or integration of the scaffold into recipient tissue. Despite the absence of a scaffold, the therapeutic effect is comparable with, or more satisfactory than conventional techniques. If desired a degradable scaffold may be used that includes or consists of one or more materials that are sensitive to defined conditions such as the pH value or the temperature of the ambience. The use of such material may allow for a controlled removal of the scaffold or parts thereof.

In some embodiments the substrate may be flexible to any extent. In such embodiments the substrate may be exposed to a change in shape upon its use as a graft, thereby adapting to geometric requirements. If the substrate includes or consists of biodegradable matter, it will slowly decompose in vivo, being largely replaced by biological matter, such as the extracellular matrix of cells used.

The term “extracellular matrix” refers to a substance existing between somatic cells such as epithelial cells. In vivo the extracellular matrix is matter located outside of cells of a multicellular organism. Extracellular matrices are typically produced by cells, and therefore, are biological materials. Extracellular matrices are involved in supporting tissue as well as in internal environmental structure essential for survival of all somatic cells. A respective matrix can also be formed or transferred in vitro and ex vivo. Extracellular matrices are generally produced from connective tissue cells. Some extracellular matrices are secreted from cells possessing basal membrane, such as epithelial cells or endothelial cells. Extracellular matrices are roughly divided into fibrous components and matrices filling there between. Fibrous components include collagen fibers and elastic fibers. A basic component of matrices is a glycosaminoglycan (acidic mucopolysaccharide), most of which is bound to non-collagenous protein to form a polymer of a proteoglycan (acidic mucopolysaccharide-protein complex). In addition, matrices include glycoproteins, such as laminin of basal membrane, microfibrils around elastic fibers, fibers, fibronectins on cell surfaces, and the like. Particularly differentiated tissue has the same basic structure. For example, in hyaline cartilage, chondroblasts characteristically produce a large amount of cartilage matrices including proteoglycans. In bones, osteoblasts produce bone matrices which cause calcification. Examples of proteins associated with an extracellular matrix, i.e. proteins found within an extracellular matrix of tissues, include, but are not limited to, elastin, vitronectin, fibronectin, laminin, collagen type I, collagen type III, collagen type V, collagen type VI, and proteoglycans (for example, decolin, byglican, fibromodulin, lumican, hyaluronic acid, etc.).

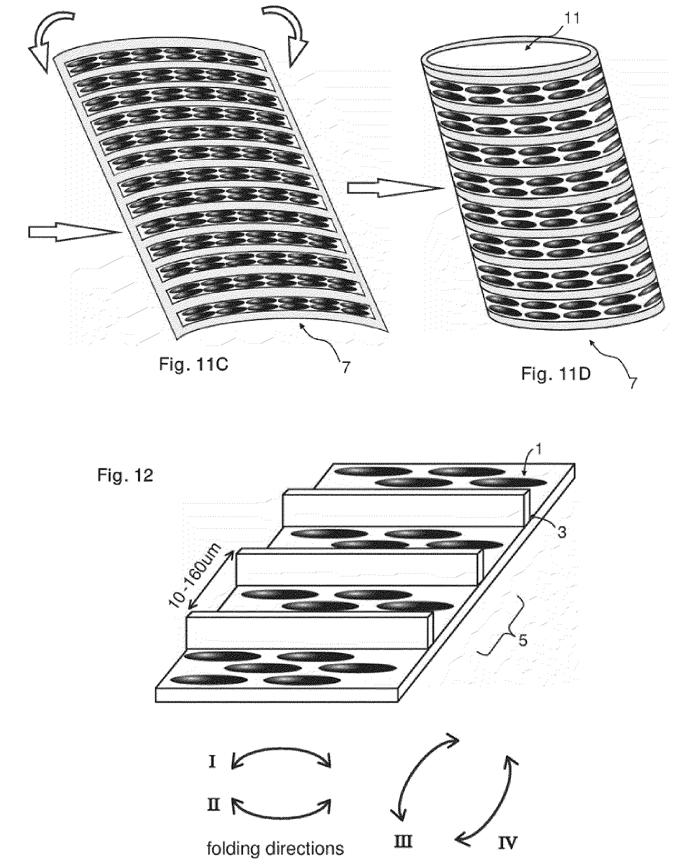

A flexible substrate may also be used to provide a desired three-dimensional shape of a graft after forming the layers of cells in the microchannels thereof. As an illustrative example, a flat substrate may be bent into a desired shape such as a tube, as illustrated in e.g. FIG. 10 or 11. Any ends of the substrate that contact each other after bending may then be connected, for example by changing the properties of the contacting surfaces, causing them to adhere (depending on the material used), or by using further matter that can act as an adhesive.

As will be apparent from the above, the present invention also relates to a method of forming a graft, such as a vascular graft. A biodegradable substrate, which may be of bendable/flexible material, with a plurality of microchannels as above, is provided. The substrate may be flat or of any other desired shape and may include a void or one or more cavities, for example. Each microchannel may have at least one common lateral wall with a further microchannel of the plurality of microchannels, the common lateral wall separating the two microchannels. By seeding a first plurality of cells in the plurality of microchannels and allowing the cells to proliferate up to at least substantial confluence, e.g. a density of at least about 80%, about 85% or about 90% (supra) at the bases of the plurality of microchannels, a first layer of aligned cells of the first plurality of cells is formed. As explained above, contact guidance cues and/or mechanical cues provided by the pair of opposing lateral walls of each microchannel cause this unidirectional alignment of the cells. On this first layer of aligned cells a second, third, fourth, fifth etc. layer of aligned cells can be formed in the same manner (see also above). An additional biodegradable material may be used to cover any or each of the cell layers thus formed before seeding a subsequent layer on a top thereof. The common lateral walls separating two microchannels of the plurality of microchannels may in some embodiments be of, or include, a biodegradable material that differs from the substrate, which may also be biodegradable. The material of the channel walls may for instance decompose at a much faster rate than the remaining substrate, or it may under selected conditions (e.g., light, oxygen, elevated temperature) degrade much faster than the substrate. In such embodiments the channel walls may be allowed to degrade to any extent—including entirely—while the remaining substrate remains at least essentially intact. It may be desired to use the structure (that includes layers of cells) in this state as a graft. In other embodiments the entire substrate, including the walls, may be degraded in vivo or in vitro, i.e. before or after using the formed structure as a graft. As explained above, if the substrate is flat, i.e. plane (in particular if it is in the form of a sheet) and of flexible matter, the method of forming a graft may include bending the substrate. Thereby a circumferential wall may for instance be formed.

A graft obtained as described above, including a vascular graft, may be used in surgery for replacing a diseased or damaged portion of a patient's vasculature. Accordingly, the present invention also provides a method of treating a patient in need of vascular prosthesis. In this regard the present invention also provides a treatment by filling, replacing and/or covering a lesion.

A vascular graft obtainable by the method of the invention may generally be used to bypass obstructions to blood flow, caused for instance by the presence of atherosclerotic plaques. It may also be used in providing arterial-venous shunts in dialysis patients, and in treating aneurysms. Tubular vascular grafts, obtainable by the method of the invention as described above, are particularly well suited for use in end-to-end anastomoses, i.e., where the damaged portion of the blood vessel is dissected and the ends of the tubular graft are connected to the cut ends of the blood vessel to span the dissected portion. A further use is in end-to-side anastomoses, i.e., where the end of a graft tube is typically attached to the side of a blood vessel. Such tubular vascular grafts are also useful in percutaneous applications, where the graft is inserted percutaneously and is positioned to span a damaged or diseased portion of a blood vessel without dissection.

### EXEMPLARY EMBODIMENTS OF THE INVENTION

Exemplary embodiments of methods according to the invention are shown in the following examples and the appending figures. It is understood that these examples are not to be considered to limit the scope of the invention, and modifications will readily occur to those skilled in the art, which modifications will be within the spirit of the invention and the scope of the appended claims.

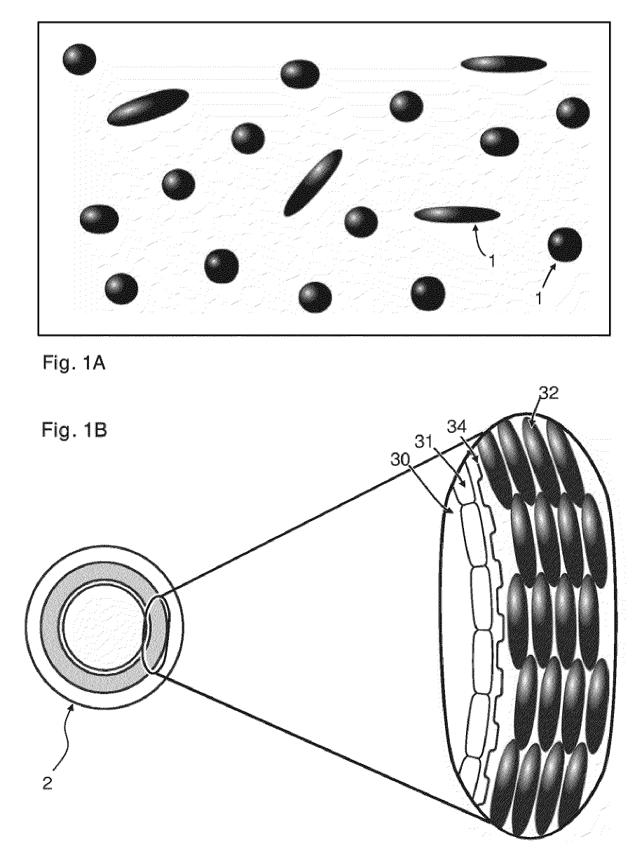

FIG. 1A depicts a conventional scaffolding technique of forming a three-dimensional structure of cells. No control on the cellular organization is provided, so that cells orientate randomly. FIG. 1B depicts on the left hand side a schematic of typical vascular tissue such as an artery. The enlargement on the right shows cells such as endothel cells (31) facing the lumen (30). Separated by the elastica interna (34), smooth muscle cells (32) form a central aligned unit of the tissue, followed by the adventitia (not enlarged), of the tissue.

FIG. 2 depicts a currently used method of forming a vascular graft by employing a pulsatile perfusion reactor (Jeong et al., 2006, supra). As depicted in FIG. 2A, into an elastic porous scaffold of tubular shape smooth muscle cells are injected. After a pre-selected period of 1, 2 or 6 h more culture medium is added. The scaffold is then connected to pulsatile perfusion bioreactor. After 2 days of static growth, a pulsatile flow (10) is applied, as depicted in FIG. 2B. Arrows indicate the direction of pulsatile flow. As a result, in the porous scaffold a radial distention force acts on the smooth muscle cells.

FIG. 3 depicts a further currently used method of obtaining unidirectionally aligned smooth muscle cells by employing a pulsatile perfusion reactor (Cha et al., 2006, supra). As depicted in FIG. 3A, a porous sheet-type substrate of polyurethane is provided. The substrate is placed in a stretching chamber and smooth muscle cells are seeded thereon. The sheet with the cells is then exposed to cyclical mechanical strain by means of a motor (the arrows indicate the stretching direction). A maximally permissible exposure time to mechanical strain of 18 h was observed. Longer incubation periods yielded cells that lacked an elongated morphology and that showed no increased actin content compared to control cells (ibid.). Furthermore, in all cases cells that had entered the pores of the substrate appeared to be of the proliferative rather than the contractile phenotype (ibid.).

FIG. 4 shows a further method of obtaining unidirectionally aligned SMCs, previously used by the present applicants (Shen et al., 2006, supra; US patent application 2007/009572 A1). A substrate with a microchannel can be provided, as depicted in FIG. 4A. SMCs are seeded thereon and grown. Confluence-triggered cell alignment occurs in the microchannel, as sketched in FIG. 4B. A substrate with a plurality of microchannels can also be provided, as depicted in FIG. 4C. Cells in each microchannel align as shown in FIG. 4D. Similar to the method depicted in FIG. 3, cells were also monolayer thick.

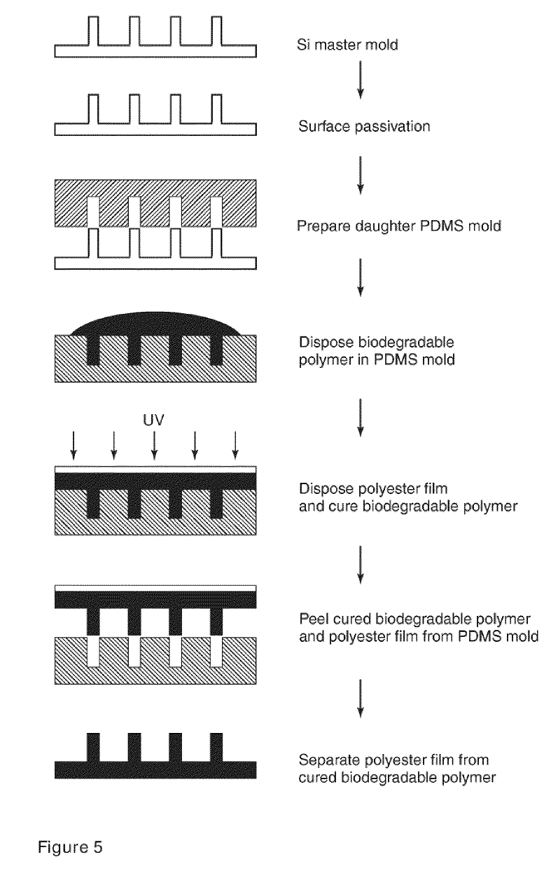

FIG. 5 shows the fabrication process of a microstructured biodegradable film using PCLLGA diacrylate. A master mold was prepared. A Teflon-like polymer was generally be used as the material for surface passivation of the master mold. A daughter mold, such as a polydimethylsiloxane (PDMS) mold, was fabricated from the master mold. In the daughter mold a biodegradable material, used for a three-dimensional biodegradable substrate, was prepared. A polyester film was disposed on the biodegradable material. The biodegradable material was cured or polymerized. Unreacted monomer was extracted from the cured biodegradable material. The cured biodegradable material and the polyester film were separated from each other leaving the cured biodegradable material for use as a substrate in the method of the invention.

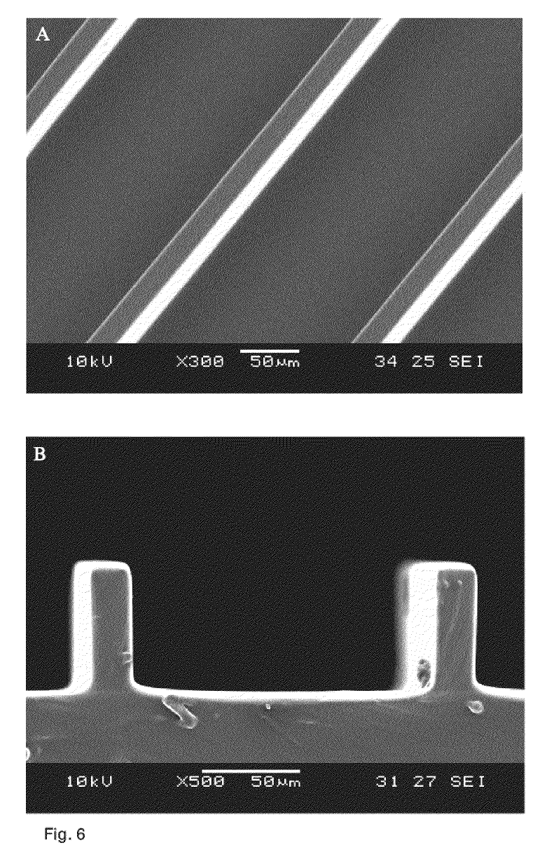

FIG. 6 shows scanning electron microscopy (SEM) images of a substrate with microchannels used in an embodiment of the invention in top view (FIG. 6A) and cross-sectional view (FIG. 6B). A common wall separating two channels is ˜25 μm wide, a channel is about 160 μm wide. The depth of a channel is 60 μm.

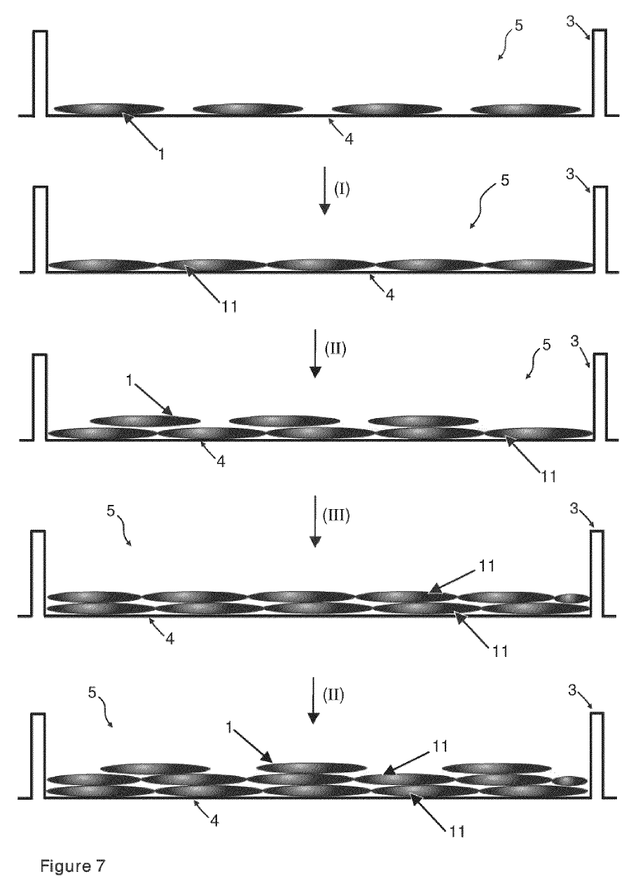

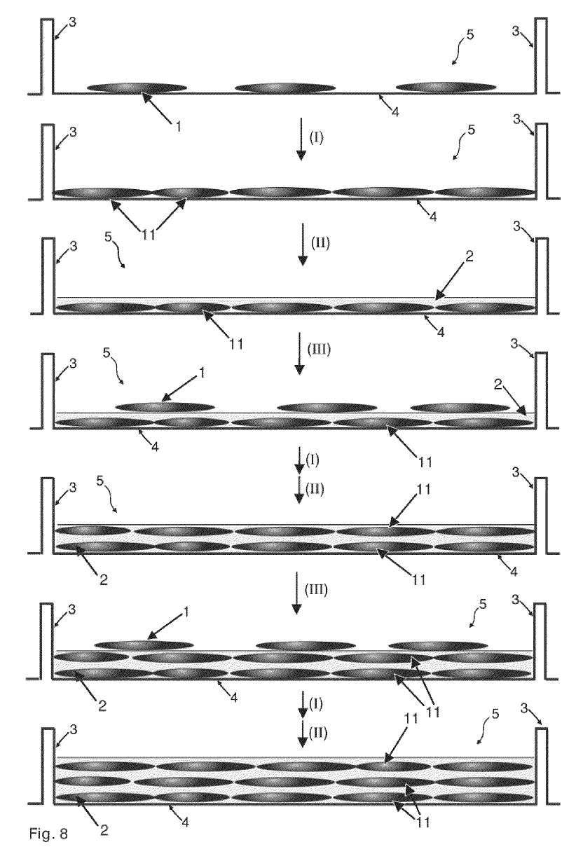

FIG. 7 depicts a schematic of a layer-by-layer (LBL) process according to the present invention, by which aligned and elongated multilayers of cells are created in microchannels of a substrate. The figure is a cross-sectional view of a microchannel along the length thereof. A first plurality of cells (1) is seeded in a microchannel (5), which is defined by at least a pair of opposing lateral walls (3) and a base (4) (I). A first cell layer (11) forms as the cells proliferate. The cells are allowed to reach confluence. A second plurality of cells (1) is seeded in the microchannel (II). A second cell layer (11) forms as the cells proliferate. The cells are allowed to reach confluence (III). A third plurality of cells (1) is seeded in the microchannel, i.e. step (II) is repeated. Steps (III) and (II) may be repeated as often as desired with a further plurality of cells. Compared to the embodiment depicted in FIG. 8, avoiding any layer of biodegradable matter that is formed on a cell layer simplifies the LBL process of the invention. Using collagen to form inter-cell-sheet layers (FIG. 8), it was also observed that layer thickness may be difficult to control both during collagen application and during its subsequent shrinkage.

FIG. 8 depicts a schematic of a further layer-by-layer (LBL) process according to the present invention. The figure is again a cross-sectional view of a microchannel along the length thereof. Similar to the method depicted in FIG. 7, aligned and elongated multilayers of cells are created in microchannels of a substrate. A first plurality of cells (1) is seeded in a microchannel (5), which is defined by at least a pair of opposing lateral walls (3) and a base (4). A first cell layer forms as the cells proliferate. The cells are allowed to reach confluence (I). A biodegradable material is added into the microchannel, where it forms a layer that covers the first cell layer (II). A second plurality of cells (1) is seeded in the microchannel (III). A second cell layer forms as the cells proliferate. These cells are, similar to the first plurality of cells, allowed to reach confluence (I), and a biodegradable material is again added into the microchannel, covering the new layer of cells (II). A further plurality of cells is seeded (III) and these steps may be repeated as often as desired, with a further plurality of cells and a further biodegradable material.

FIG. 9 depicts an embodiment of a method of the invention using a substrate (6), depicted in FIG. 9A, with a circumferential wall. The circumferential wall is of circular profile. A plurality of microchannels (5) is arranged on the circumferential wall. Each microchannel shares a common wall (3) with a further microchannel and is separated form the further microchannel by the wall (3). A layer-by-layer process according to the present invention as depicted in FIG. 7 or FIG. 8 is carried out on the plurality of microchannels. Multiple layers of unidirectionally aligned cells (1) are thereby formed as shown in FIG. 9B. Where the substrate is of tubular or cylindrical shape, a vascular graft is thereby formed. At least for embodiments in which a substrate of cylindrical shape is used, an in vitro degradation, at least to a certain extent, of the substrate may be desired.

FIG. 10 depicts a further embodiment of forming a vascular graft using the method of the present invention. As shown in FIG. 10A a flat substrate (7) of biodegradable material is provided. The substrate is of bendable material. A plurality of microchannels (5) is arranged on the surface of the flat substrate. Each microchannel shares at least one common wall (3) with a further microchannel and is separated form the respective further microchannel by the wall (3). A layer-by-layer process according to the present invention as depicted in FIG. 7 or FIG. 8 is carried out on the plurality of microchannels. Multiple layers of unidirectionally aligned cells (1) are thereby formed as shown in FIG. 10B (only the top layer of cells is delineated for sake of clarity). The flat substrate is carefully bent (FIG. 10C), so that a circumferential wall with a lumen (11) is formed, as shown in FIG. 10D.

FIG. 11 depicts an embodiment of forming a vascular graft that resembles the embodiment depicted in FIG. 10. On a flat substrate (7) of biodegradable, bendable material a plurality of microchannels (5) with common walls (3) is arranged. The microchannels are however oriented perpendicular to the microchannels shown if FIG. 10. A layer-by-layer process according to the present invention as depicted in FIG. 7 or FIG. 8 is carried out on the plurality of microchannels. Multiple layers of unidirectionally aligned cells (1) are thereby formed as shown in FIG. 11B. Similar to FIG. 10, the flat substrate (7) is carefully bent (FIG. 10C), so that a circumferential wall with a lumen (11) is formed (FIG. 10D). Contrary to the embodiment depicted in FIG. 10, the substrate (7) is bent along the direction that at least roughly corresponds to the longitudinal axis of the microchannels (5). Accordingly, the microchannels run at least approximately along (or only slightly inclined to) the circumference of the tube of the formed vascular graft. In contrast thereto, the microchannels in the embodiment depicted in FIG. 10D run in a direction perpendicular thereto. In the embodiment depicted in FIG. 10D the longitudinal axis of each microchannel is at least approximately parallel to the longitudinal axis of the tube of the formed vascular graft. While the embodiment shown in FIG. 10 is suitable for endothelial cells, which need to aligned in a circumferential direction around of the tube of the formed vascular graft, the embodiment shown in FIG. 11 is suitable for smooth muscle cells, which need to aligned in the longitudinal direction of the tube. In this regard it is noted that endothelial cells are to be arranged on the luminal side, so that the substrate (7) will be bent accordingly (opposite to the direction shown in FIG. 10C).

FIG. 12 illustrates the growth of cells (1) in microchannels (5) on a flat substrate of biodegradable, bendable material in a close-up view. Individual microchannels share common walls (3), by which they are separated form each other. Depending on the desired purpose (which also determines the type of cell used), the substrate may be bent in any direction, the most common directions being shown in the figure, labeled I to IV. Directions I and II bend the longitudinal axis of the microchannels, thereby forcing them into a circular shape. Directions III and IV bend the lateral axis of the microchannels, leaving them essentially unchanged in the longitudinal dimension.

FIG. 13 depicts schematically a folded tubular structure suitable for smooth muscle cells (not shown for the sake of clarity). The microchannels (5), separated by walls (3), revolve around the central lumen (11). Since the substrate is flexible, the tubular structure is bendable and flexible as well and can adapt to geometrical requirements of its surroundings.

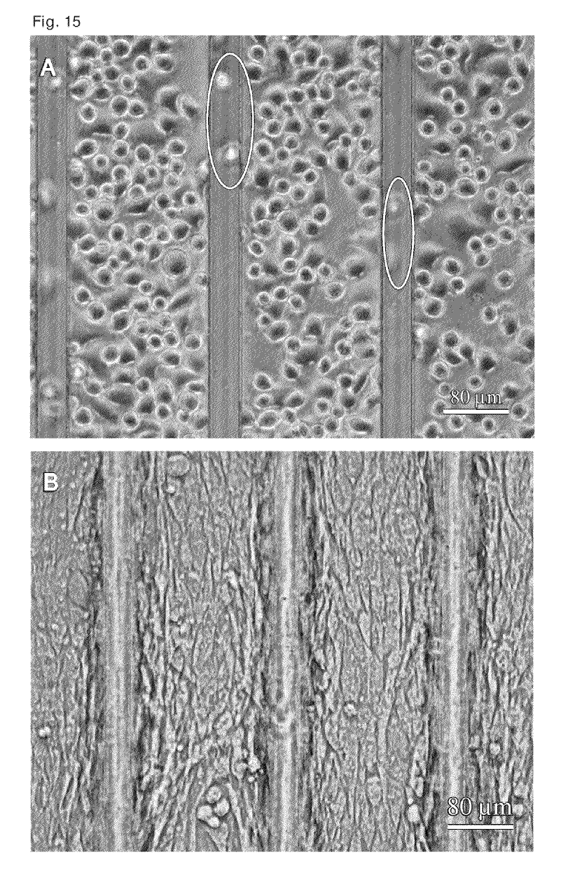

FIG. 14 depicts the correlation between the seeding density of smooth muscle cells and the time needed for the cells to reach confluence in microchannels. With higher initial seeding densities less time was required for the cells to become confluent, align and to undergo a morphology change. At a seeding density of 2×10⁵ cells/cm², cell confluence, cell alignment and substantial elongation had already occurred after 0.5 days.

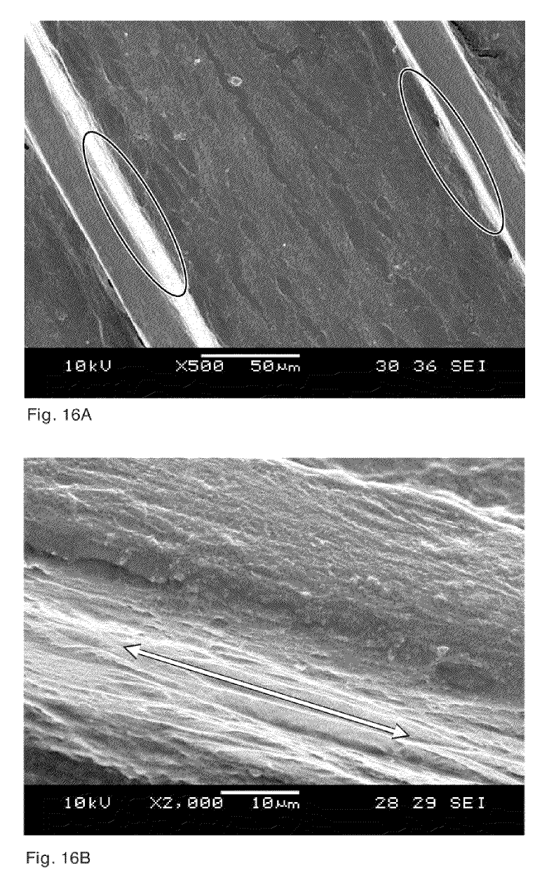

FIG. 15 depicts optical microscopy images of the formation of a confluent layer of smooth muscle cells in a microchannel in a method according to the present invention. Cells were seeded at 2×10⁵ cells/cm², so that a further plurality of cells could already be seeded after 0.5 days. FIG. 15A shows the cells at the beginning of culture, while FIG. 15B shows the cells having reached confluence within 0.5 days. All the seeded smooth muscle cells, which appeared round on seeding, attached to the substrate surface, elongated and then aligned along the microchannel direction quickly.

Notably smooth muscle cells seeded onto the substrate with microchannels rather than individually into each microchannel not only dropped into channel bottoms but also onto the wall ridges (FIG. 15A, circled region). This effect did not derogate the obtained three-dimensional structure of unidirectionally aligned cells in any way. Furthermore, after reaching confluence, the two sides of each lateral channel wall were also covered by aligned/elongated smooth muscle cells. Scanning by confocal microscope in the z-direction from wall ridge to channel bottom confirmed this phenomenon (not shown). This demonstrated that a layer of well aligned and elongated smooth muscle cells, formed in the method of the invention, provides a continuous cell sheet, such that cells in adjacent microchannels have cell-cell communication.

FIG. 16A shows scanning electron microscopy images of smooth muscle cells, grown on a substrate with microchannels used in an embodiment of the invention for seven days, in 500—(A) and 2000-fold magnification (B). The cells not only aligned on bottom of microchannels, ridges of microwalls, but also on the two vertical sides of the microwalls. Cell initial seeding density was 5×10⁴ cells/cm².

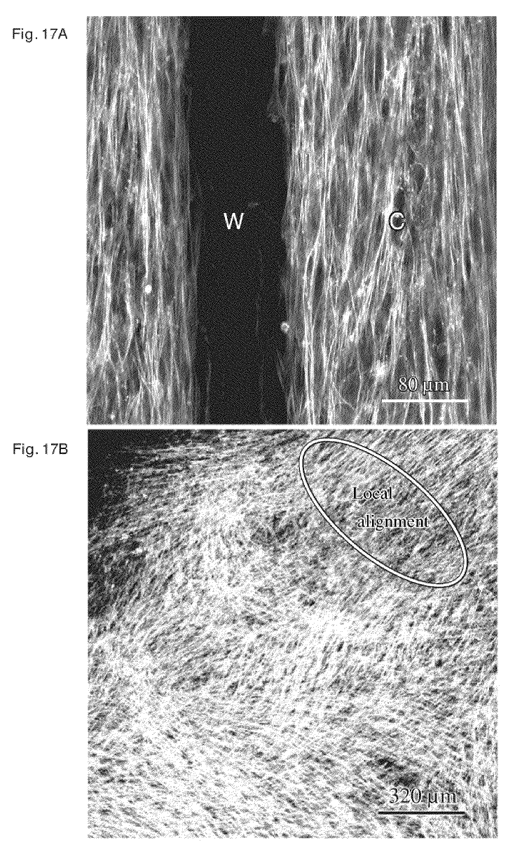

FIG. 17A depicts F-actin-stained smooth muscle cells on a substrate that included a plurality of microchannels, 6 days after seeding at a density of 2×10⁵ cells/cm² (for rapid alignment and elongation, see FIG. 14 and FIG. 15). The lateral wall of the channel is denoted by “w”, and the recessed region formed by the channel is denoted by “c”. As can be seen in FIG. 17A, F-actin filament fluorescence imaging demonstrated that a rapidly aligned cell state achieved within 0.5 days is a state that can be maintained for long periods of time rather than a transient effect. For comparison, FIG. 17B depicts F-actin-stained smooth muscle cells on a uniformly flat control surface after 6 days of monolayer culture, also seeded at 2×10⁵ cells/cm². FIG. 17B is shown in low magnification in order to present a larger area. As can be seen, F-actin filaments of smooth muscle cells cultured on a uniformly flat surface are randomly orientated. Nevertheless local patches of uni-directionally aligned cells can spontaneously form due to their natural near-neighbor interactions (circled region in FIG. 17B). However, such local alignment typically occurs over an area of no more than 1 mm². Over larger areas no uni-directionally alignment of smooth muscle cells can be observed. Thus such a cell layer lacks the organization desired for vascular function.

FIG. 18 depicts F-actin-stained smooth muscle cells seeded and grown on microchannels of a substrate according to the method of the invention (four layers of cells), as depicted in FIG. 8. “W” indicates the location of the lateral walls of the microchannels, while “C” indicates a microchannel, in which the cells were grown. Cells are spaced with a thin layer of a collagen gel. The construct was scanned with a scanning step of 4 mm from the channel bottom to the top surface (A-D). The images are optic sections representative of continuous first, second, third, and fourth layers of the three dimensional structure, each spaced by 8 mm. The dark band in the microchannel part of image C may be a section of gel layer caused by inhomogeneous overcoating. Cell seeding density for each layer was 1.5×10⁵ cells/cm².

FIG. 19A depicts alpha-actin-stained smooth muscle cells, seeded and grown on microchannels of a substrate according to the present invention. As can be seen, the cells patterned in the microchannels (and thus constrained at confluence by the channel walls) appeared elongated and had actin filaments oriented along the longitudinal direction of the channels. FIG. 19B depicts alpha-actin-stained smooth muscle cells on a gel layer overfilled above the top of the microchannel walls. Accordingly, these cells grew above the channel walls and lacked the respective contact guidance cues. As a result these cells grew in a nonoriented manner. These results illustrate the effect of contact guidance provided by the microchannels, which can be used to obtain a three dimensional pattern of aligned cells, when applying the LBL process of the invention. The optical depth of both photos is 12 mm.

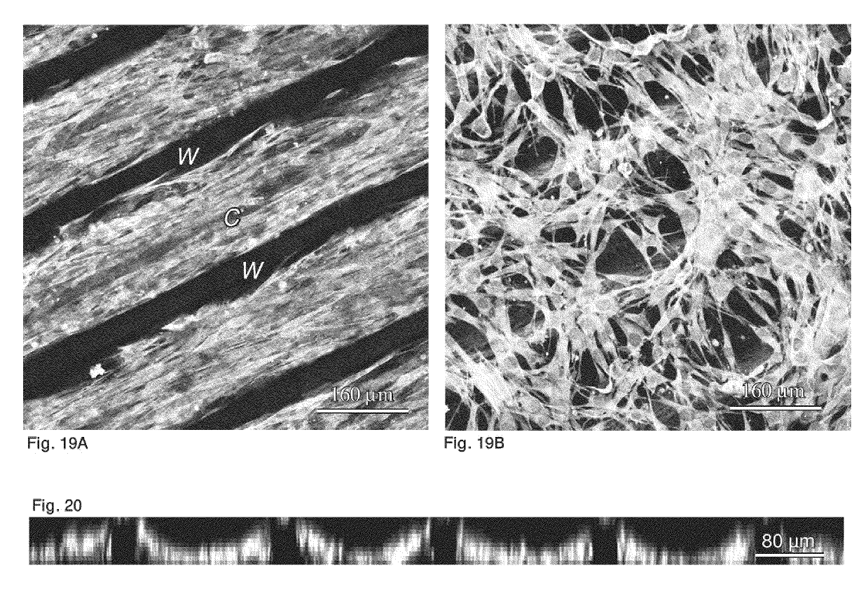

FIG. 20 depicts a cross-sectional image of four layers of F-actin-stained smooth muscle cells, obtained by the method of the present invention. The layers of aligned smooth muscle cells are interleaved with collagen (cf. FIG. 8). The image was obtained by scanning in the x-z plane, using confocal microscopy. The cell layers are bow-shaped. In theory, the thickness of SMC multilayers in different microchannels should be statistically similar. The obtained data of the depicted sample showed an average thickness of the multilayers at the lowest point (i.e., at the center of the microchannel) of 24 mm, the calculated variation in thickness between individual microchannels was about 1.3 mm.

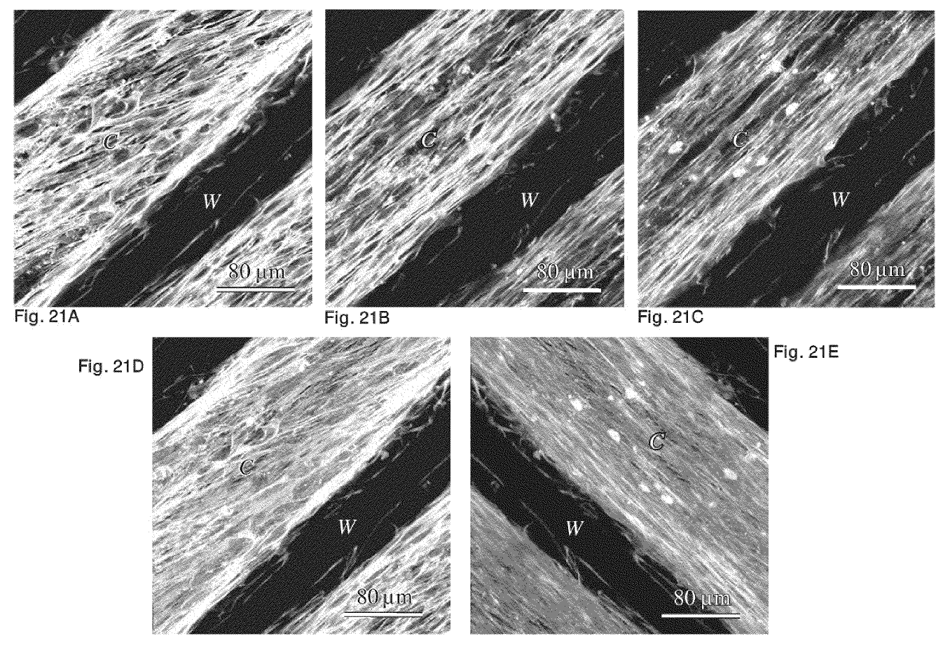

FIG. 21 depicts F-actin-stained smooth muscle cells, seeded and grown on microchannels of a substrate according to the present invention. Five layers of cells were grown as depicted in FIG. 7, without an interleaving collagen gel. The obtained structure was mounted upside down on a microslide and scanned with a scanning step of 2 mm from the top to the bottom of a channel (A-C) by fluorescence confocal imaging. Images A to C are optic sections representative of the fifth, the third, and first layers of the three dimensional structure, spaced by 10 mm. (D) and (E) are 3D composite images of A, B, and C, viewed from the top to the bottom of a channel and vice versa. Cell seeding density for each layer was 1.5×10⁵ cells/cm². Similar to the smooth muscle cells separated by collagen, the smooth muscle cells appeared aligned and elongated through the depth of the microchannel in the absence of collagen. In fact, the distribution of cells within each cell plane (first, third, and fifth layers in the structure of five layers) also seemed more homogenous than that of corresponding cells spaced by collagen gel layers. Noteworthy, omitting collagen layers not only simplified the LBL process, thereby accelerating the speed of cell patterning, but also provided an additional native-like topological cue for the alignment and elongation of newly seeded cell layers. Moreover, it enhances the important cell-cell interactions between adjacent cell layers.

### Materials

Polydimethylsiloxane (PDMS) (Sylgard 184, Dow Corning, MI) was used as received. Poly(ε-caprolactone-r-L-lactide-r-glycolide) (PCLLGA) diacrylate was synthesized in our lab (Shen et al., 2006, supra). Unless otherwise stated, other chemicals were of reagent grade and bought from Sigma (St. Louis, Mo.) and used as received.

### Methods

Fabrication of a microchanneled scaffold: A substrate with microchannels was fabricated by UV embossing of a 4-inch p-type silicon wafer (Shen et al., 2006, supra).

Macromer synthesis: Poly(ε-caprolactone-r-L-lactide-r-glycolide) (PCLLGA) diacrylate was prepared by the ring opening polymerization of ε-caprolactone, L-lactide, and glycolide with tetra(ethylene glycol) and stannous octoate as the initiator and catalyst, respectively. The polymer was designed with a molecular weight of 9,300; the ratio of CL/LA/GA was 60:20:20. Synthesis procedures were as follows: 0.59 g (0.003 M) tetra(ethylene glycol), 4.32 g L-lactide, 3.48 g glycolide, and 20.52 g ε-caprolactone, and stannous octoate ( 1/1,000 of the total weight) were added into a 100-mL round-bottomed flask equipped with a stirring bar and high vacuum stopcock, which was connected to a dual bank manifold with one end connected to vacuum pump and the other to argon gas. Polymerization was carried out under stirring for 24 h at 145° C. under argon atmosphere after 3 purging cycles with argon gas. The reaction mixture was cooled to room temperature, precipitated in heptane and diethyl ether, and dried at 45° C. under reduced temperature to give a clear viscous liquid. The isolated polymer was dissolved in dichloromethane (100 mL/10 g solid) in a 3-neck round-bottomed flask and was cooled to 0° C. in an ice bath. 1.75 g (0.019 M) acryloyl chloride and 1.95 g (0.019 M) triethylamine dissolved in dichloromethane (20 mL) were dropwise added into the flask. The mixture was reacted at 0° C. for 6 h and then at room temperature for 18 h. Dichloromethane was then removed by rotary evaporation and the yield was precipitated in diethyl ether twice to remove the excess acryloyl chloride and triethylamine. After that, the viscous oil was dissolved in tetrahydrofuran and triethylamine hydrochloride was allowed to precipitate in the solution for 24 h. Then the solution was filtrated and tetrahydrofuran was removed by rotary evaporation. The viscous oil was then precipitated in ethanol twice to remove the remaining triethyamine hydrochloride and precipitate in diethyl ether to afford diacrylated polymers. The excess diethyl ether was removed under reduced pressure at 65° C. for 24 h. The obtained macromers were clear light yellow viscous liquid.

The diacrylated polymer was characterized by nuclear magnetic resonance (¹H-NMR) on a Bruker DMX-300 spectrometer (Billerica, Mass.) at 300 MHz using CDCl₃ as a solvent, Fourier transform infrared (FTIR) spectroscopy on a Nicolet 560 spectrometer over the wavenumber range 4,000-400 cm⁻¹, gel permeation chromatograph on an Agilent 1000 differential refractometer HPLC system (Agilent, Palo Alto, Calif.) using tetrahydrofuran as eluent at a flow rate of 1.0 mL/min, and differential scanning calorimetry (DSC) (TA DSC 2920 Modulated DSC) running double cycles from −80 to 80° C. with a heating rate of 20° C./min and cooling rate of 10° C./min under nitrogen atmosphere.

Fabrication of microstructured polymeric films: The fabrication of a microstructured biodegradable film is diagramed in FIG. 5. A master Si mold was first prepared using a surface technology system deep reactive ion etching (DRIE) according to a published procedure. Additionally, the microstructured Si (4-inch <100> p-type silicon wafers) master mold was surface treated with a passivation step to deposit a Teflon-like polymer on it, which is critical for clean demolding. C4F8 was used for the passivation using the DRIE system. The plasma power, C4F8 flow rate, and pressure in the chamber were 300 W, 100 sccm, and 26 mTorr, respectively, and the duration was 90 sec. The master mold has 10 groups of microstructures, each of which is 60-70 mm deep. Five groups of the microstructures have microwalls that are 10 mm wide; the remaining are 25 mm wide. All microwalls are 2 cm long. The microchannels between them were either 10, 40, 80, 120, or 160 mm wide. For ease of reference, the microstructure geometries are hereafter denoted as w/c where w is width of the microwall and c is width of microchannel.

Silastic J RTV (Dow Corning Corporation, Midland, Mich.) was poured on the silicon master mold to form flexible and reusable child PDMS molds from which UV embossing was performed. Biodegradable PCLLGA diacrylate was stirred for 2 h at 65° C. with 0.5 wt % photoinitiator 2,2-dimethoxy-2-phenylacetophenone (Irgacure 651 photoinitiator; CIBA Chemicals, Basel, Switzerland) predissolved in butanone (10% Irgacure 651 in butanone); the excess butanone was removed under reduced pressure at 65° C. for 2 h. The UV resin formulation was dispensed onto the PDMS mold and allowed to spread in an oven set at a temperature of 65° C. Following this, degassing in a vacuum oven at a pressure of 0 atmosphere and a temperature of 65° C. was carried out to remove air bubbles and promote resin formulation filling of the mold microchannel.

A polyester film (Melinex 454, DuPont Teijin Films [Hopewell, Va.], 125 mm thick) was then carefully overlaid onto the resin to avoid the formation of any air bubbles. The polyester substrate was chosen to have marginal adhesion to PCLLGA so that the PCLLGA would adhere to the substrate more strongly than to the PDMS or Si mold, but could be easily peeled from it after demolding. Finally, the resin was polymerized under 365-nm UV for 10 min. The patterned films were then carefully peeled from the PDMS mold and the micropatterned film was lifted up from the polyester film. The UV source was a flood UV exposure system with an Hg-lamp, specifically a 350-W mercury lamp with intensity of 10 mW/cm² at 365 nm of a mask aligner system (SUSS MicroTech, Bremen, Germany). An elastic PCLLGA film with deep microchannels (having a depth of 60 μm) separated by narrow microwalls (width of 25 μm) was obtained (FIG. 6). Such deep and wide (160 μm) microchannels provide mechanical constraints needed for SMC alignment/elongation and sufficient space for the construction of a three-dimensional tissue, resembling the SMC medial layer of arteries. For ease of describing the width of microwalls (w) and microchannels (c), they are herein labelled w/c; for example, 25/160 refers to microchannels which are 160 μm wide separated by 25 μm wide microwalls. Much narrower (e.g. 25/40) and wider microchannels (e.g. 25/300 or 25/500) had also been used in our earlier studies. But narrow channels have been observed to limit cellular proliferation and wider channels do not promote unidirectional alignment and elongation; thus narrow and wide channels would not be adopted in the study here (Shen et al., 2006, supra).

SMC culture: Microchanneled films were cut into 1.5 cm diameter discs, sterilized and then moved to a 24-well culture plate. SMCs (ATCC, CRL-1444) from rat aorta suspended in complete growth medium (Dulbecco's Modified Eagle's Medium (DMEM) with 4 mM L-glutamine supplemented with 10% fetal bovine serum, 50 IU/ml of penicillin and 50 μl/ml of streptomycin) were added to the plate wells at different initial seeding densities (varying from 1×10⁴ to 2×10⁵ cells/cm²). Cell cultures were maintained in a humidified 95% air-5% CO₂ incubator at 37° C. Medium was refreshed every 2 days before confluence and every one day after confluence. SMC morphology (specifically alignment and elongation) at different culture durations were observed and imaged with a ZEISS inverted microscope.

LBL 3D fabrication: LBL 3D fabrication was performed by successive application of our optimized 2D SMC patterning (all with initial seeding density of 1.5×10⁵ cells/cm²). Two LBL approaches were employed. The first method involved application of a thin collagen Type I gel layer to each SMC layer after it neared confluence in order to provide a substrate for the seeding of the next SMC layer. The 2^{nd} method dispensed away the gel layer and directly seeded high density SMCs on the prior confluent layers (denoted as No Gel process). For the former method, the time interval between successive SMCs seeding is 1.5 days; 1 day was needed for cell to reach confluence and 0.5 day for collagen gelation and further contraction (24). For the latter method, the time interval between successive cell layers is 1 day. The collagen gel employed in the “interleaved” LBL process was prepared by quickly mixing 500 μl DMEM+400 μl collagen Type I solution (4 mg/ml in 0.2% acetic acid)+50 μl 10×PBS+50 μl 10×DMEM+10 μl 1 N NaOH at 0-4° C. (25). After aspiration off the culture medium from the first confluent layer, 25 μl of collagen pre-gel solution was placed onto the culture film. After removal of excess pre-gel solution, gelation at 37° C. for 20 min and contraction further for 0.5 day, the next SMC layer was seeded. Repetition of this cycle resulted in a 3D multilayered structure of SMC sheets within the scaffold microchannels (FIG. 8).

SMCs visualization: Cell orientation within the microchannels was characterized by imaging F-actin and α-actin filaments after immunostaining. For F-actin staining, 3D SMC cultures were washed with phosphate buffered saline (PBS) 3 times, fixed in 3.7% formaldehyde for 10 mins and permeabilized with 0.5% Triton X-100 for 5 mins at room temperature. After blocking with 1% bovine serum albumin (BSA) solution for 30 mins to minimize background signal, samples were incubated with 5 μl 4 U/ml rhodamine-conjugated phalloidin (Molecular Probes) for 20 mins. For α-actin staining, blocked samples were incubated with monoclonal anti-smooth muscle α-actin (Sigma, 1:75) for 1 h. After 3 thorough washings with PBS, 5 μl 300U Alexa Fluor® 488 (Molecular Probes) in 200 μl 1% BSA solution was added and samples were incubated for another 1 h. After washing with PBS 3 times, samples were mounted and used for fluorescence confocal microscopy (LSM510 META, Carl Zeiss, Germany). To examine the cultures three-dimensionally, serial optical sections which were made by scanning the F-actin stained multilayers in Z-direction with a scanning step of 2 or 4 μm were further reconstructed into 3D images by LSM510 META software.

Cross-section scanning of F-actin stained multilayers (4 layers interleaved with collagen gel) in the x-z plane was also performed to measure the thickness variation between multilayers in different microchannels. 20 microchannels in 3 batches of scanning at different locations were selected and the thickness of multilayers in each channel at the lowest height point was measured.

In earlier studies on 2D patterning of SMCs (Shen et al., 2006, supra; US patent application 2007/009572 A1), the present applicants found that with initial seeding densities as low as 1×10⁴ cells/cm², SMCs cultured in microchannels (with widths varying from 10 to 160 μm) could grow to confluence with aligned/elongated morphology along the direction of the microchannels after 7 days. Cultures on flat films did not exhibit such large-scale parallel alignment and elongation. The confluence induced SMC alignment and elongation was achieved with the simple conditions of culture to confluence in the presence of a physical growth barrier. We postulate that the confluence-induced 2D alignment and elongation can be extended to SMC 3D patterning by a layer-by-layer process. 2D culture is useful for fundamental studies but 3D culture is necessary for re-creation of functional organ and tissue substitutes. However, repeats of the 7-day culture cycle needed for layer-by-layer 3D patterning will take too long for the fabrication of a native-like blood vessel for clinical application in an acute care setting.

Since the unidirectional alignment and elongation are induced by constrained confluence, it is postulated that the speed at which 2D elongation and alignment are triggered are influenced by the initial seeding density. Support for this hypothesis can be found in FIG. 14, which shows that higher initial seeding density resulted in a shorter time until confluence was reached and alignment and cell morphology change occurred. At a seeding density of 2×10⁵ cells/cm², 0.5 days were sufficient for SMC confluence as well as alignment and substantial elongation. With the microchannels, the SMCs which appear round on seeding all attached, elongated and then aligned along the microchannel direction quickly (FIG. 15). Cells grown without microchannels will also attach and elongate but they will not be unidirectionally aligned. Moreover, F-actin filament fluorescence imaging demonstrated that such a rapidly aligned state could be maintained for long periods of at least 6 days during the culture of subsequent layers (FIG. 17A) (“w” means wall and “c” means channel in all the figures). F-actin filaments of SMCs cultured on unpatterned control film are randomly orientated (FIG. 17B). The durability of the SMC alignment and the ability to quickly culture subsequent aligned sheets implies that the whole period needed for 3D patterning of a “thick” (by the standards of small vessel engineering) SMC tissue by the LBL process could be markedly shortened, e.g., 10 layers could be rapidly fabricated in 5 days. The implications for clinical application are apparent.

As a sidenote, SMCs seeded on microchanneled film did not only fall onto channel bottoms but also onto the wall ridges (FIG. 15A, circled region). Moreover, after reaching confluence, the two sides of each wall were also covered by aligned/elongated SMCs. Scanning by means of a confocal microscope in the z-direction from wall ridge to channel bottom confirmed this phenomenon (see FIG. 17). This demonstrated that well aligned/elongated SMCs monolayer induced by microchanneled substrate is a continuous cell sheet so that cells in adjacent microchannels have cell-cell communication.

Based on the data obtained for the 2D patterning process, 4-layer assembly of SMCs on the microchanneled film by the method of the invention was investigated. Thin inter-cell-sheet collagen gel layers were applied as illustrated in FIG. 8. Serial optical sections of fluorescently dyed F-actin filament made by confocal scanning in the Z-direction showed different cell layers, each of which defined by elongated SMCs, highly aligned along the longitudinal axis of the channels (FIG. 18). Staining of smooth muscle α-actin also showed that SMCs patterned in the microchannels (and thus constrained at confluence by the channel walls) appeared elongated and had actin filaments oriented along the direction of the channels (FIG. 19A). While SMCs that were above the channel walls due to overfilling of the collagen pre-gel grew in a non-oriented manner as they were not contained within the channels of the scaffold (FIG. 19B). These results demonstrate that a deeply microchanneled substrate can be used as a scaffold to form a three-dimensionally patterned cell configuration by the LBL process of the present invention.

Cell viability assessment: In the LBL process, mass transport in terms of nutrient supply to and waste removal from the bottom layer is important. Mass transport in terms of nutrient supply to, and waste removal from the inside, is a noteworthy issue for any 3D tissues. A simple live/dead cell assay was performed by Trypan Blue staining to address this topic. Dead cells would take up the dye and appear blue. SMCs after monolayer culture for 6 days and LBL culture (as illustrated in FIG. 7, i.e. without layering a gel between cells) with 4 layers (with interval of 1.5 days between cell cycles, all cells initially seeded at 1.5×10⁵ cells/cm²) were detached by trypsin-EDTA solution and prepared into cell suspension by addition of DMEM (the cell concentration was adjusted to 5˜20×10⁵ cells/ml). 50 μl of such cell suspension was mixed thoroughly with 50 μl of 0.4% Trypan Blue and allowed to sit for 2-3 minutes. Then approximately 9 μl of the Trypan Blue/cell suspension mixture was added into hemacytometer counting chambers to observe under optical microscope. The stained cells were first counted and then the total cells. Cell viability was calculated as the ratio of number of unstained (living) cells to total cells counted.

The results showed that not only monolayer SMCs possessed high cell viability (96.6%) after 6-day culture but also those in 4-layer No Gel LBL culture after 6 days (1.5 days per cycle) also showed high viability (95.4%), demonstrating that mass transport, as well as the metabolism of SMCs in multilayers, is active in our 4-layer LBL 3D construct.

In summary, the LBL process avoids the “steric hindrance” effect and exploits the advantages of 2D cell patterning in the deep microchannel, specifically the ability to topologically influence cell alignment and morphology. Although SMCs cultured on flat surfaces can align and elongate to form local patches of uni-directionally aligned cells due to their natural near-neighbor interactions (FIG. 17B, circled region), such local alignment typically occurs over no more than 1 mm² area; over larger areas the SMCs are not uni-directionally aligned and lack the organization desired for vascular function. With our LBL fabricated 3D structure reported here, the unidirectional orientation of F-actin and α-actin filaments would potentially lead to higher tensile and contractile strength along the microchannel direction. In native vessels, both F-actin and α-actin contribute towards the medial layer strength and vasoactivity. A three-dimensional microchanneled sheet, which produces desirable 2D cell alignment and morphology, can be combined with the LBL process of the invention to produce thick SMC tissues with high tensile strength in one direction.

To achieve a final “tissue-like” homogenously aligned SMC multilayer, the relatively narrow microwalls should be degraded away, either in vitro, or partially in vivo. Ideally, the mechanical properties and biodegradability of the microwall should be adjusted to match the SMC growth rate. The degradation and mechanical properties of the PCLLGA copolymer can be adjusted by varying the oligomer molecular weight and composition. Typically, the lower e-CL content, the faster the degradation; the lower the oligomer molecular weight, the slower the degradation. Biodegradable aliphatic polyesters based on caprolactone, glycolide acid and lactide acid have been widely used in tissue engineering and implants such as sutures because their degradation products are cytocompatible. For example, elastic poly(L-lactide-co-ε-caprolactone) (PLCL, 50:50) scaffold has been reported to exhibit excellent tissue compatibility to SMCs and might be very useful for vascular tissue engineering. Moreover, these polymers have gained the approval of the US Food and Drug Administration (FDA) for human clinical use in a variety of applications.

SMCs and other cells that align may also be proliferated, aligned and oriented in circumferential microchannels of a tubular scaffold, in particular on the outside thereof. This requires a special tubular mold for the micropatterning (see e.g. FIG. 9 or FIG. 13). The aligned cells, e.g. SMCs on the microchanneled tubular scaffold would potentially provide an excellent prototype tissue engineered medial layer with improved burst strength and vasoactivity similar to that found in native blood vessels. Alternately, the LBL process could also be performed on microchanneled film and wrapped into a tubular form towards fabricating much thicker vascular medial layer rapidly (see e.g. FIGS. 10-12). Cell-cell interaction between different wrapping layers can be realized by fabricating porous or hydrogel microchanneled scaffolds.

In practical vascular tissue engineering, the 3D tissue of SMC on microchanneled scaffold must be finally combined with endothelial cell (EC) lining and even a fibroblast tissue layer. Lining the lumen of synthetic vascular prostheses with a thin layer of endothelial cell is a good method to avoid thrombogenicity. In many studies already being performed on SMC/EC co-culture, researchers mostly used micron or sub-micron porous membrane (unpatterned) as double-side substrate for promoting communication between the two kinds of cells on opposite sides of the membrane. The method of the present invention can be used for this purpose, e.g. using a porous microchanneled scaffold. The microchannels can for instance be fabricated on the outside of a tubular porous scaffold, while EC layer lines can be formed facing the lumen of the tube. Such kind of design would not only benefit fixing of the transplanted tube by the fibroblast ingrowth into channels in vivo, but also easy supplying of a thromboresistant layer.

The listing or discussion of a previously published document in this specification should not necessarily be taken as an acknowledgement that the document is part of the state of the art or is common general knowledge. All documents listed are hereby incorporated herein by reference in their entirety for all purposes.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Additional objects, advantages, and features of this invention will become apparent to those skilled in the art upon examination of the foregoing examples and the appended claims. Thus, it should be understood that although the present invention is specifically disclosed by exemplary embodiments and optional features, modification and variation of the inventions embodied therein herein disclosed may be resorted to by those skilled in the art, and that such modifications and variations are considered to be within the scope of this invention. In addition, where features or aspects of the invention are described in terms of Markush groups, those skilled in the art will recognize that the invention is also thereby described in terms of any individual member or subgroup of members of the Markush group.

What is claimed is:

1. A method of forming a three-dimensional structure of unidirectionally aligned cells, the method comprising:
(a) providing a substrate with a microchannel, wherein said microchannel is defined by at least a pair of opposing lateral walls and a base, and wherein in at least a portion of the microchannel the distance between the pair of opposing lateral walls is within the micrometer range,
(b) seeding a first plurality of cells in the microchannel,
(c) allowing cells of the first plurality of cells to proliferate up to at least substantial confluence at the base of the microchannel, thereby (i) providing contact guidance cues by the pair of opposing lateral walls of the microchannel, such that the cells align unidirectionally, and (ii) forming a first layer of aligned cells of the first plurality of cells at the base of the microchannel,
(d) seeding a second plurality of cells in the microchannel, wherein the base of the microchannel already comprises a first layer of aligned cells of the first plurality of cells, and
(e) allowing cells of the second plurality of cells to proliferate up to at least substantial confluence at the base of the microchannel, thereby (i) providing contact guidance cues by the lateral walls of the microchannel, such that the cells align unidirectionally, and (ii) forming a second layer of aligned cells of the second plurality of cells at the base of the microchannel.

2. The method of claim 1, wherein the microchannel has a longitudinal axis, and wherein the contact guidance by the pair of opposing lateral walls is along the longitudinal axis of the microchannel.

3. The method of claim 1, wherein at least a portion of the base of the microchannel has an at least essentially straight surface.

4. The method of claim 3, wherein the portion of the base of the microchannel is the portion in which the distance between the two opposing lateral walls is from about 10 microns to about 600 microns.

5. The method of claim 1, wherein the distance between the two opposing lateral walls of the microchannel is selected from the group consisting of a distance from about 10 microns to about 600 microns and a distance from about 60 microns to about 300 microns.

6. The method of claim 1, wherein step (d) comprises seeding the second plurality of cells onto the first layer of aligned cells of the first plurality of cells at the base of the microchannel, such that the second plurality of cells contacts the first plurality of cells in the microchannel.

7. The method of claim 1, wherein step (d) further comprises:
covering the first layer of the aligned cells of the first plurality of cells at the base of the microchannel with a biodegradable material.

8. The method of claim 7, wherein the second plurality of cells is seeded on the biodegradable material.

9. The method of claim 7, wherein the biodegradable material forms a hydrogel.

10. The method of claim 7, wherein the biodegradable material is collagen.

11. The method of claim 1, wherein the microchannel has an aspect ratio of its depth to its width from about 0.1 to about 50.

12. The method of claim 1, wherein the microchannel has a depth from about 10 microns to about 500 microns.

13. The method of claim 1, wherein at least the portion of the substrate that comprises the microchannel comprises a biodegradable material.

14. The method of claim 13, wherein the biodegradable material is selected from the group consisting of a polyglycolide, a polylactide, a polycaprolactone, a polyamide, an aliphatic polyester, a poly(ester amide), a poly(amino acid), a pseudo-poly(amino acid), a poly(lactide glycolide), poly(lactic acid ethylene glycol), poly(ethylene glycol), poly(ethylene glycol) diacrylate, a polyalkylene succinate, polybutylene diglycolate, polyhydroxybutyrate, polyhydroxyvalerate, a polyhydroxybutyrate/polyhydroxyvalerate copolymer, poly(hydroxybutyrate-co-valerate), a polyhydroxyalkaoates, a poly(caprolactone-polyethylene glycol)copolymer, poly(valerolactone), a polyanhydride, a poly(orthoester), a polyanhydride, a polyanhydride ester, poly(anhydride-co-imide), an aliphatic polycarbonate, a poly(hydroxyl-ester), a polydioxanone, a polycyanoacrylate, a poly(alkyl cyanoacrylate), a poly(amino acid), a poly(phosphazene), a poly(propylene fumarate), poly(propylene fumarate-co-ethylene glycol), a poly(fumarate anhydride), a poly(propylene carbonate), fibrinogen, fibrin, gelatin, cellulose, a cellulose derivative, chitosan, alginate, a polysaccharide, starch, amylase, collagen, a polycarboxylic acid, a poly(ethyl ester-co-carboxylate carbonate), poly(iminocarbonate), poly(bisphenol A-iminocarbonate), poly(trimethylene carbonate), poly(ethylene oxide), poly(epsilon-caprolactone-dimethyltrimethylene carbonate), a poly(alkylene oxalate), a poly(alkylcarbonate), poly(adipic anhydride), a nylon copolyamide, carboxymethyl cellulose, a copoly(ether-ester), a polyether, a polyester, a polydihydropyran, a polyketal, a polydepsipeptide, a polyarylate, a poly(propylene fumarate-co-ethylene glycol), a hyaluronates, poly-p-dioxanone, a polyphosphoester, a polyphosphoester urethane, a polysaccharide, starch, rayon, rayon triacetate, latex, and a composite thereof.

15. The method of claim 1, further comprising allowing the contact guidance cues provided by the lateral walls of the microchannel to cause the cells of the first layer of unidirectionally aligned cells and/or the second layer of unidirectionally aligned cells to show an elongated morphology.

16. The method of claim 1, wherein the first plurality of cells and the second plurality of cells comprise cells of the same cell type.

17. The method of claim 1, wherein the first plurality of cells and/or the second plurality of cells are selected from the group consisting of smooth muscle cells, skeletal muscle cells, myocytes, fibroblasts, endothelial cells, bone marrow cells, neurons, pericytes and epithelial cells.

18. The method of claim 1, wherein the cells of the first plurality of cells and/or the second plurality of cells are human cells.

19. The method of claim 1, wherein the substrate comprises a plurality of microchannels, each channel being defined by at least a pair of opposing lateral walls and a base, wherein in at least a portion of each microchannel the distance between the two opposing lateral walls is from about 10 microns to about 600 microns.

20. The method of claim 19, wherein at least two microchannels of the plurality of microchannels are separated by a common wall.

21. A method of forming a vascular graft, the method comprising:
(a) providing a biodegradable substrate with a plurality of microchannels, wherein each of said plurality of microchannels is defined by at least a pair of opposing lateral walls and a base, wherein in at least a portion of each microchannel the distance between the pair of opposing lateral walls is within the micrometer range, and wherein each microchannel has at least one common lateral wall with a further microchannel of said plurality of microchannels, the common lateral wall separating the two microchannels,
(b) seeding a first plurality of cells in the plurality of microchannels,
(c) allowing cells of the first plurality of cells to proliferate up to at least substantial confluence at the bases of the plurality of microchannels, thereby (i) providing contact guidance cues by the pair of opposing lateral walls of each microchannel, such that the cells align unidirectionally, and (ii) forming a first layer of aligned cells of the first plurality of cells at the base of each microchannel,
(d) seeding a second plurality of cells in each microchannel, wherein the base of each microchannel already comprises a first layer of aligned cells of the first plurality of cells, and
(e) allowing cells of the second plurality of cells to proliferate up to at least substantial confluence at the base of the microchannel, thereby (i) providing contact guidance cues by the pair of opposing lateral walls of each microchannel, such that the cells align unidirectionally, and (ii) forming a second layer of aligned cells of the second plurality of cells at the base of each microchannel.

22. The method of claim 21, further comprising:
allowing the biodegradable substrate, including the common wall separating two microchannels of the plurality of microchannels, to be degraded.

23. The method of claim 21, wherein the substrate is flat and of bendable material.

24. The method of claim 23, further comprising bending the substrate, thereby forming a circumferential wall.

25. A method of treating a patient in need of vascular prosthesis, the method comprising replacing a diseased or damaged portion of the patient's vasculature with the vascular graft obtained according to the method of claim 21.
